(19) United States
(12) Patent Application Publication
(10) Pub. No.: US 2024/0240145 A1
(43) Pub. Date: Jul. 18, 2024

(54) METHODS AND COMPOSITIONS FOR GENERATING MITOCHONDRIA REPLACED LYMPHOID CELLS

(71) Applicant: Imel Biotherapeutics, Inc., Waltham, MA (US)

(72) Inventors: Satoshi GOJO, Kyoto-Fu (JP); Daisuke KAMI, Kyoto-Fu (JP)

(21) Appl. No.: 18/562,815

(22) PCT Filed: May 18, 2022
(86) PCT No.: PCT/IB2022/054639

§ 371 Date: Nov. 20, 2023

Related U.S. Application Data
(60) Provisional application No. 63/190,078, filed on May 18, 2021.

(51) Int. Cl. C12N 5/0783; A61K 35/17; A61P 37/02; C12M 1/26

(52) U.S. Cl. CPC C12N 5/0636; A61K 35/17; A61P 37/02; C12M 33/10; C12N 2501/999

(57) ABSTRACT

The present disclosure provides methods and compositions for generating mitochondria replaced lymphoid cells, such as T cells, that involves incubating lymphoid cells that have not undergone a procedure to reduce or deplete endogenous mitochondria with isolated exogenous mitochondria and an effective amount of mammalian target of rapamycin (mTOR) inhibitor. In addition, the present disclosure also provides methods of treating an immunological deficiency associated with heteroplasmic immune cells, as well as methods for ameliorating a symptom of mitochondrial complex III deficiency, that involve administering the mitochondria replaced lymphoid cells.

This application claims the benefit of U.S. Provisional Application No. 63/190,078, filed May 18, 2021, which is incorporated herein by reference in its entirety.

### 1. SEQUENCE LISTING

The instant application contains a Sequence Listing which is submitted electronically in ASCII format and is hereby incorporated by reference in its entirety. Said ASCII copy, created on May 18, 2022, is named 14595-008-228_SL.txt and is 3,681 bytes in size.

### 2. INTRODUCTION

The present disclosure relates, in part, to methods and compositions for generating mitochondria replaced lymphoid cells. In a specific aspect, the disclosure relates to methods for generating mitochondrial replaced lymphoid cells (e.g., T cells) without prior mitochondrial deletion or depletion using a composition comprising rapamycin and equivalents thereof, as well as therapeutic methods for treating diseases using such mitochondrial replaced lymphoid cells. The disease that may be treated using the mitochondrial replaced lymphoid cells include not only diseases caused by inherited and acquired mitochondrial DNA mutations, such as mitochondrial diseases, but also immunological deficiencies associated with heteroplasmic immune cells.

### 3. BACKGROUND

Mitochondrial dysfunction can arise from various factors, such as genetic disorders. In certain circumstances, mitochondrial diseases or disorders can negatively impact the function of lymphocytes. For example, mitochondrial complex III is involved in the suppressive function of regulatory T cells (Tregs), and mitochondrial complex III deficiency can impair Treg function. Mitochondrial dysfunction can also arise as a result of genotoxic agents, aging, oxidative stress inflammation, and/or injury. For example, exposure to therapeutic agents (e.g., nucleoside/nucleotide reverse transcriptase inhibitors) can reduce mitochondrial DNA (mtDNA) copy number. Mitochondrial transfer from a healthy cell to a damaged or dysfunctional cell can help to rescue and restore mitochondria function.

Current methods of transferring mitochondria to a recipient cell include methods that involve partial or complete depletion of endogenous mtDNA. For example, the classical method to remove endogenous mtDNA involves long term treatment of cells with low concentrations of ethidium bromide (EtBr), a known carcinogen and teratogen, limiting its application for therapeutic purposes. Additional techniques to transfer mitochondria include methods that use invasive instruments, which are harmful to the recipient cell and inefficient.

Cell based therapies involving lymphocytes have emerged as a means to fight disease and alleviate medical conditions. Often there are a limited number of lymphocytes available for such therapies. Consequently, any modifications to the cells must not only be safe for administration to the patient, but also gentle enough so as to not damage the lymphocytes. Thus, there is a significant unmet need to develop safe and effective methods for mitochondrial transfer in lymphocytes, including in cell-based therapy settings that involve lymphocytes.

### 4. SUMMARY

In one aspect, provided herein is a method for generating mitochondria replaced lymphoid cells, wherein the method comprises incubating lymphoid cells that have not undergone a procedure to reduce or deplete endogenous mitochondria with isolated exogenous mitochondria and an effective amount of mammalian target of rapamycin (mTOR) inhibitor for a sufficient period of time to non-invasively transfer the exogenous mitochondria to the lymphoid cells, thereby generating mitochondria replaced lymphoid cells in which at least 20% of the endogenous mtDNA has been replaced with exogenous mtDNA. In a specific embodiment, provided herein is a method for generating mitochondria replaced lymphoid cells in which at least 20% of endogenous mitochondrial DNA (mtDNA) has been replaced with exogenous mtDNA, wherein the method comprises incubating lymphoid cells that have not undergone a procedure to reduce or deplete endogenous mitochondria with isolated exogenous mitochondria and an effective amount of mammalian target of rapamycin (mTOR) inhibitor for a sufficient period of time to non-invasively transfer the exogenous mitochondria to the lymphoid cells, thereby generating mitochondria replaced lymphoid cells in which at least 20% of the endogenous mtDNA has been replaced with exogenous mtDNA. In one embodiment, the mTOR inhibitor includes rapamycin or a derivative thereof. In a specific embodiment, the mTOR inhibitor is rapamycin. In certain embodiments, the effective amount of the mTOR inhibitor is a concentration of about 100 nM to about 1000 nM. In some embodiments, the effective amount of the mTOR inhibitor is a concentration of about 200 nM to about 500 nM. In one embodiment, the effective amount of the mTOR inhibitor is a concentration of about 100 nM. In another embodiment, the effective amount of the mTOR inhibitor is a concentration of about 200 nM. In another embodiment, the effective amount of the mTOR inhibitor is a concentration of about 500 nM. In another embodiment, the effective amount of the mTOR inhibitor is a concentration of about 1000 nM. In specific embodiments, the mitochondria replaced lymphoid cells includes at least 20% of exogenous mtDNA and no more than 80% endogenous mtDNA, as measured by TaqMan Single Nucleotide Polymorphism (SNP) Assay.

In specific embodiment, provided herein is a method for generating mitochondria replaced lymphoid cells, wherein the method comprises incubating lymphoid cells that have not undergone a procedure to reduce or deplete endogenous mitochondria with isolated exogenous mitochondria and about 100 nM to about 1000 nM of rapamycin for a sufficient period of time to non-invasively transfer the exogenous mitochondria to the lymphoid cells, thereby generating a mitochondria replaced lymphoid cells.

In some embodiments, the methods provided herein include isolated exogenous mitochondria that is about 20 μg to 80 μg protein per 1×10⁶ cells. In certain embodiments, the methods provided herein further comprise centrifuging the lymphoid cells prior to incubating. In one embodiment, centrifuging is performed at 1,500 relative centrifugal force (RCF) for approximately 5 minutes at room temperature.

In some embodiments, the sufficient period of time for the methods provided herein is at least approximately 24 hours. In certain embodiments, the sufficient period of time for the methods provided herein is at least 36 hours. In some embodiments, the sufficient period of time for the methods provided herein is at least 48 hours. In certain embodiments, the sufficient period of time for the methods provided herein is approximately 2 days or more. In some embodiments, the sufficient period of time for the methods provided herein is approximately 7 days or more. In one embodiment, the sufficient period of time for the methods provided herein is approximately 2 days to approximately 7 days.

In yet another aspect, provided herein is a method for generating mitochondria replaced lymphoid cells, wherein the method comprises: (a) centrifuging lymphoid cells and isolated exogenous mitochondria under conditions sufficient to generate a cell pellet, wherein the lymphoid cells have not undergone a procedure to reduce or deplete endogenous mitochondria; and (b) incubating the lymphoid cells with 100 nM to 1000 nM of rapamycin for approximately 24 hours or more, thereby generating mitochondria replaced lymphoid cells.

In certain embodiments, incubating is for approximately 7 days or more. In some embodiments, incubating is for approximately 2 days to approximately 7 days. In some embodiments, the lymphoid cells of the methods provided herein are T cells, B cells, monocytes, macrophages, natural killer (NK) cells, or granulocytes. In certain embodiments, the lymphoid cells of the methods provided herein are T cells. In some embodiments, the T cells comprise exhausted T cells, senescent T cells, or a combination thereof. In certain embodiments, the lymphoid cells of the methods provided herein are lymphoid cells are human lymphoid cells.

In another aspect, provided herein are mitochondria replaced lymphoid cells generated by the methods described herein, and compositions comprising such cells.

In another aspect, provided herein is a composition comprising an effective amount of the mitochondria replaced lymphoid cells of the present disclosure, and a pharmaceutically acceptable carrier.

In another aspect, provided herein is a method for ameliorating a symptom of mitochondrial complex III deficiency to a subject in need thereof, comprises administering to the subject the composition that includes an effective amount of the mitochondria replaced lymphoid cells of the present disclosure, and a pharmaceutically acceptable carrier. In a specific embodiment, the subject is human.

In another an aspect, provided herein is a method for treating an immunological deficiency associated with heteroplasmic immune cells to a subject in need thereof, that includes administering to the subject the composition that includes an effective amount of the mitochondria replaced lymphoid cells of the present disclosure, and a pharmaceutically acceptable carrier. In a specific embodiment, the subject has received a reverse transcriptase inhibitor. In certain embodiments, the subject has human immunodeficiency virus (HIV). In some embodiments, the subject has hepatitis B virus (HBV). In a specific embodiment, the subject is human.

### 5. BRIEF DESCRIPTION OF THE FIGURES

FIG. 1. Mitochondrial DNA (mtDNA) Sequences. MtDNA sequences isolated from human GJ T cells and EPC100 cells were sequenced and compared. Differences in mtDNA sequences were detected by sequencing the D Loop, and some differences were observed in the D Loop hypervariable region 1 (“HVR1”).

FIGS. 2A-2B. Primers and Probes for SNP Assay. FIG. 2A shows the sets of primers (SEQ ID NOs: 5 and 6) and probes (SEQ ID NOs: 1 and 2) for the SNP assay for detecting differences in the HVR1 of human mtDNA from human GJ T cells and EPC100 cells. FIG. 2B depicts where the primers (SEQ ID NOs: 5 and 6) and probes (SEQ ID NOs: 1 and 2) bind to the HVR1 of human mtDNA from human GJ T cells and EPC100 cells.

FIGS. 3A-3B. FIG. 3A depicts the protocol for transfer of isolated mitochondria from EPC100 cells to human GJ T cells. FIG. 3B shows the results of SNP assays to detect the replacement of GJ T cell mtDNA with mtDNA from EPC100 cells.

FIGS. 4A-4B. FIG. 4A depicts the protocol for transfer of mitochondria from B6 mouse embryonic fibroblasts (MEF) cells to mouse NZB T cells. FIG. 4B shows the depiction of the titration of rapamycin in the wells.

FIG. 5A-5B. FIG. 5A depicts the results of SNP assay on day 2. FIG. 5B depicts the results of SNP assay on day 7. Orange is the ratio of the endogenous mitochondrial genotype of NZB T cell and blue is the ratio of the exogenous mitochondrial genotype from B6 MEF cells.

### 6. DETAILED DESCRIPTION

Provided herein are methods for generating mitochondria replaced lymphoid cells that involve incubating lymphoid cells, which have not undergone a procedure to reduce or deplete endogenous mitochondria, with isolated exogenous mitochondria and an effective amount of mammalian target of rapamycin (mTOR) inhibitor. The mitochondria replaced lymphoid cells generated according to the methods disclosed herein have utility as a therapy, such as for ameliorating a symptom of mitochondrial complex III deficiency or immunological deficiency associated with heteroplasmic immune cells.

As used herein, the term “mitochondria replaced lymphoid cell” is generally intended to mean a lymphoid cell in which endogenous mitochondria and/or endogenous mtDNA have been substituted with exogenous mitochondria and/or exogenous mtDNA. In certain embodiments, a mitochondria replaced lymphoid cell has all the endogenous mitochondria and/or endogenous mtDNA in a lymphoid cell replaced by exogenous mitochondria and/or exogenous mtDNA. In specific embodiments, a mitochondria replaced lymphoid cell has endogenous mitochondria replaced with exogenous mitochondria. In such circumstances, the replacement of endogenous mitochondria with exogenous mitochondria is assessed by assessing mtDNA markers. In specific embodiments, a mitochondria replaced lymphoid cell has a certain percentage of endogenous mtDNA replaced with exogenous mtDNA. In some embodiments, a mitochondria replaced lymphoid cell has about 5% or more, about 10% or more, about 20% or more, about 30% or more, about 40% or more, about 50% or more, about 60% or more, about 70% or more, about 80% or more, about 90% or more, or about 95% or more of the endogenous mitochondria and/or endogenous mtDNA in a lymphoid cell replaced with exogenous mitochondria and/or exogenous mtDNA. In certain embodiments, a mitochondria replaced lymphoid cell has about 5% to about 10%, about 10% to about 20%, about 10% to about 30%, about 10% to about 40%, about 20% to about 40%, about 25% to about 50%, about 25% to about 75%, about 50% to about 75%, about 40% to about 50%, about 75% or more to about 85%, about 75% to about 95% of the endogenous mitochondria and/or endogenous mtDNA in a lymphoid cell replaced with exogenous mitochondria and/or exogenous mtDNA. In some embodiments, a mitochondria replaced lymphoid cell has at least 5%, at least 10%, at least 15%, at least 20%, at least 30%, at least 40%, at least 50%, at least 60%, at least 70%, at least 80%, at least 90%, or at least 95% of the endogenous mitochondria and/or endogenous mtDNA in a lymphoid cell replaced with exogenous mitochondria and/or exogenous mtDNA.

As used herein, the term “isolated” when used in reference to mitochondria generally refers to mitochondria that have been physically separated or removed from the other cellular components of its natural biological environment. In a specific embodiment, a technique described in the Example, infra, is used to isolate mitochondria.

As used herein, the term “isolated” when used in reference to a cell generally means a cell that is substantially free of at least one component as the referenced cell is found in nature. The term includes a cell that is removed from some or all components as it is found in its natural environment. The term also includes a cell that is removed from at least one, some or all components as the cell is found in non-naturally occurring environments. Therefore, an isolated cell is partly or completely separated from other substances as it is found in nature or as it is grown, stored or subsisted in non-naturally occurring environments. Specific examples of isolated cells include partially pure cells (e.g., lymphoid cells), and substantially pure cells (e.g., lymphoid cells) that are enriched from other cell types (e.g., non-lymphoid cells). Accordingly, a referenced cell that is isolated may be 50%, 60%, 70%, 80%, 90%, 95%, 99%, or 100% pure of other cells and/or substances. In a specific embodiment, a technique described in the Example, infra, is used to isolate the referenced cell.

As used herein, the term “exogenous” is understood by the skilled person in the art. Generally, the term “exogenous” refers to cellular material (e.g., mitochondria or mtDNA) that is not from the recipient cell. For example, exogenous mitochondria or mtDNA may be isolated from a fibroblast introduced into a T cell, such as described in the Example, infra.

As used herein, the term “endogenous” is generally understood by the person skilled in the art. Generally the term “endogenous” refers to cellular material (e.g., mitochondria or mtDNA) that is native to the recipient cell.

As used herein, the term “effective amount” generally refers to the amount of a compound or composition necessary to achieve the desired result(s) under the relevant conditions.

As used herein, the terms “about” or “approximately” when used in conjunction with a number generally refer to any number within 1, 5, 10, 15 or 20% of the referenced number as well as the referenced number.

As used herein, the term “sufficient period of time” generally refers to an amount of time that produces the desired result(s).

As used herein, the term “non-invasively” when used in reference to the transfer of exogenous material (e.g., mitochondria and/or mtDNA) is generally intended to mean without the use of invasive instruments (e.g., nanoblade or electroporation), or harmful conditions that compromise the structure of the cell.

As used herein, the term “subject” is generally intended to mean an animal. A subject can be a human or a non-human mammal, such as a dog, cat, bovid, equine, mouse, rat, rabbit, or transgenic species thereof. It is understood that a “subject” can also refer to a “patient,” such as a human patient.

The terms “heteroplasmy” and “heteroplasmic” are understood by the skilled person in the art. Generally, the terms “heteroplasmy” and “heteroplasmic” refer to the occurrence of more than one type of mtDNA genome in an individual or sample.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, M D (1999); Glover, ed., DNA Cloning, Volumes I and II (1985); Gait, ed., Oligonucleotide Synthesis (1984); Hames & Higgins, eds., Nucleic Acid Hybridization (1984); Hames & Higgins, eds., Transcription and Translation (1984); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and Weir & Blackwell, eds., Handbook of Experimental Immunology, Volumes I-IV (1986).

### 6.1 Methods of Generating a Mitochondria Replaced Cell (MirC)

The present disclosure is based, in part, on the finding that the use of an mTOR inhibitor can enhance the transfer of donor mitochondria to a recipient lymphocyte without the need for any prior reduction or depletion of the recipient cell's endogenous mitochondria and/or endogenous mitochondrial DNA (mtDNA). Accordingly, in one aspect, provided herein are methods for generating mitochondria replaced lymphoid cells using an mTOR inhibitor without the use of any procedure for prior reduction or depletion of endogenous mitochondria and/or endogenous mtDNA. In a specific embodiment, provided herein is a method for generating mitochondria replaced lymphoid cells, comprising incubating lymphoid cells, which have not undergone a procedure to reduce or deplete endogenous mitochondria, with isolated exogenous mitochondria and an effective amount of mammalian target of rapamycin (mTOR) inhibitor for a sufficient period of time to non-invasively transfer the exogenous mitochondria to the lymphoid cells, thereby generating mitochondria replaced lymphoid cells. In another specific embodiment, provided herein is a method for generating mitochondria replaced lymphoid cells, comprising incubating lymphoid cells, which have not undergone a procedure to reduce or deplete endogenous mitochondria, with isolated exogenous mitochondria and a composition comprising an effective amount of mammalian target of rapamycin (mTOR) inhibitor for a sufficient period of time to non-invasively transfer the exogenous mitochondria to the lymphoid cells, thereby generating mitochondria replaced lymphoid cells. For example, the lymphoid cells that have not undergone a procedure to reduce or deplete endogenous mitochondria would not have not been contacted with a mitochondrial DNA (mtDNA) depleting agent (e.g., ethidium bromide (EtBr), or an enzyme capable of degrading mtDNA, such as a restriction enzyme), or otherwise transfected or transduced with a polynucleotide encoding a mtDNA depleting agent. In some embodiments, the lymphoid cells that have not undergone a procedure to reduce or deplete endogenous mitochondria would not have been contacted with an enzyme capable of degrading mtDNA, or transfected or transduced with a polynucleotide encoding an enzyme capable of degrading mtDNA. In certain embodiments, the methods provided herein for generating a mitochondria replaced lymphoid cell are performed in vitro or ex vivo.

The level of endogenous mtDNA that is replaced accordingly to the methods provided herein need not result in a complete replacement of endogenous mtDNA with exogenous mtDNA (i.e., 100% replacement). For example, in some embodiments, at least 10% of the endogenous mtDNA has been replaced with exogenous mtDNA. In some embodiments, at least 15% of the endogenous mtDNA has been replaced with exogenous mtDNA. In some embodiments, at least 20% of the endogenous mtDNA has been replaced with exogenous mtDNA. In some embodiments, at least 25% of the endogenous mtDNA has been replaced with exogenous mtDNA. In some embodiments, at least 30% of the endogenous mtDNA has been replaced with exogenous mtDNA. In some embodiments, at least 35% of the endogenous mtDNA has been replaced with exogenous mtDNA. In some embodiments, at least 40% of the endogenous mtDNA has been replaced with exogenous mtDNA. In some embodiments, at least 45% of the endogenous mtDNA has been replaced with exogenous mtDNA. In some embodiments, at least 50% of the endogenous mtDNA has been replaced with exogenous mtDNA. In some embodiments, at least 55% of the endogenous mtDNA has been replaced with exogenous mtDNA. In some embodiments, at least 60% of the endogenous mtDNA has been replaced with exogenous mtDNA. In some embodiments, at least 65% of the endogenous mtDNA has been replaced with exogenous mtDNA. In some embodiments, at least 70% of the endogenous mtDNA has been replaced with exogenous mtDNA. In some embodiments, at least 80% of the endogenous mtDNA has been replaced with exogenous mtDNA. In some embodiments, at least 90% of the endogenous mtDNA has been replaced with exogenous mtDNA. In some embodiments, 100% of the endogenous mtDNA has been replaced with exogenous mtDNA. Techniques known to one of skill in the art or described herein (e.g., in the Example) may be used to measure the replacement of endogenous mtDNA with exogenous mtDNA.

In a specific embodiment, provided herein is a method for generating mitochondria replaced lymphoid cells in which at least 20% of the endogenous mtDNA has been replaced with exogenous mtDNA, comprising incubating lymphoid cells, which have not undergone a procedure to reduce or deplete endogenous mitochondria with isolated exogenous mitochondria, with an effective amount of mammalian target of rapamycin (mTOR) inhibitor for a sufficient period of time to non-invasively transfer the exogenous mitochondria to the lymphoid cells, thereby generating mitochondria replaced lymphoid cells in which at least 20% of the endogenous mtDNA has been replaced with exogenous mtDNA.

Techniques known to one of skill in the art or described herein (e.g., in the Example) may be used to measure the replacement of endogenous mtDNA with exogenous mtDNA. For example, various sequencing methods can be used in combination with any of the methods provided herein to evaluate or confirm transfer of exogenous mitochondria and/or exogenous mtDNA, or quantify heteroplasmy. Generally, differences in mtDNA can be detected by sequencing the D-Loop of mtDNA. The D-Loop contains two regions within which mutations accumulate more frequently than anywhere else in the mitochondrial genome. The regions are called hypervariable regions (HVR)-1 and HVR2, respectively.

In some embodiments, endogenous mtDNA and exogenous mtDNA are sequenced and quantified in connection with the methods provided herein, by sequencing the HV1 and/or HV2 of the D-loop of mtDNA. In specific embodiments, the sequencing method comprises a single nucleotide polymorphism (SNP) assay. In other embodiments, the sequencing method comprises digital PCR. In specific embodiments, the digital PCR is droplet digital PCR.

Generally, an effective amount of the mTOR inhibitor is a concentration of about 60 nanomolar (nM) to about 1000 nM. However, the amount of the mTOR inhibitor that is effective to transfer mitochondria to the lymphocytes may be influenced by factors that include cell density, time of treatment, type of mTOR inhibitor, and type of lymphocyte.

In some embodiments, the effective amount of the mTOR inhibitor is a concentration of about 60 nM to about 1000 nM. In some embodiments, the effective amount of the mTOR inhibitor is a concentration of about 100 nM to about 1000 nM. In some embodiments, the effective amount of the mTOR inhibitor is a concentration of about 200 nM to about 500 nM. In some embodiments, the effective amount of the mTOR inhibitor is a concentration of about 300 nM to about 600 nM. In some embodiments, the effective amount of the mTOR inhibitor is a concentration of about 400 nM to about 700 nM.

In certain embodiments, the effective amount of the mTOR inhibitor is a concentration of about 60 nM. In certain embodiments, the effective amount of the mTOR inhibitor is a concentration of about 70 nM. In certain embodiments, the effective amount of the mTOR inhibitor is a concentration of about 80 nM. In certain embodiments, the effective amount of the mTOR inhibitor is a concentration of about 90 nM. In certain embodiments, the effective amount of the mTOR inhibitor is a concentration of about 100 nM. In some embodiments, the effective amount of the mTOR inhibitor is a concentration of about 150 nM. In some embodiments, the effective amount of the mTOR inhibitor is a concentration of about 200 nM. In some embodiments, the effective amount of the mTOR inhibitor is a concentration of about 250 nM. In some embodiments, the effective amount of the mTOR inhibitor is a concentration of about 300 nM. In some embodiments, the effective amount of the mTOR inhibitor is a concentration of about 350 nM. In some embodiments, the effective amount of the mTOR inhibitor is a concentration of about 400 nM. In some embodiments, the effective amount of the mTOR inhibitor is a concentration of about 450 nM. In some embodiments, the effective amount of the mTOR inhibitor is a concentration of about 500 nM. In some embodiments, the effective amount of the mTOR inhibitor is a concentration of about 550 nM. In some embodiments, the effective amount of the mTOR inhibitor is a concentration of about 600 nM. In some embodiments, the effective amount of the mTOR inhibitor is a concentration of about 650 nM. In some embodiments, the effective amount of the mTOR inhibitor is a concentration of about 700 nM. In some embodiments, the effective amount of the mTOR inhibitor is a concentration of about 750 nM. In some embodiments, the effective amount of the mTOR inhibitor is a concentration of about 800 nM. In some embodiments, the effective amount of the mTOR inhibitor is a concentration of about 850 nM. In some embodiments, the effective amount of the mTOR inhibitor is a concentration of about 900 nM. In some embodiments, the effective amount of the mTOR inhibitor is a concentration of about 1000 nM. In some embodiments, the effective amount of the mTOR inhibitor is a concentration greater than 1000 nM.

Various inhibitors of mTOR can be used to generate mitochondria replaced lymphoid cells according to the present disclosure, such as the exemplary mTOR inhibitors described in Section 6.2. In certain embodiments, the mTOR inhibitor is rapamycin or a derivative thereof. In a specific embodiment, the mTOR inhibitor is rapamycin.

In some embodiments, the mTOR inhibitor is rapamycin or a derivative thereof at a concentration of about 100 nanomolar (nM) to about 1000 nM. In some embodiments, the mTOR inhibitor is rapamycin or a derivative thereof at a concentration of about 200 nM to about 500 nM. In some embodiments, the mTOR inhibitor is rapamycin or a derivative thereof at a concentration of about 300 nM to about 600 nM. In some embodiments, the mTOR inhibitor is rapamycin or a derivative thereof at a concentration of about 400 nM to about 700 nM.

In certain embodiments, the effective amount of rapamycin or a derivative thereof is a concentration of about 100 nM. In some embodiments, the effective amount of rapamycin or a derivative thereof is a concentration of about 150 nM. In some embodiments, the effective amount of rapamycin or a derivative thereof is a concentration of about 200 nM. In some embodiments, the effective amount of rapamycin or a derivative thereof is a concentration of about 250 nM. In some embodiments, the effective amount of rapamycin or a derivative thereof is a concentration of about 300 nM. In some embodiments, the effective amount of rapamycin or a derivative thereof is a concentration of about 350 nM. In some embodiments, the effective amount of rapamycin or a derivative thereof is a concentration of about 400 nM. In some embodiments, the effective amount of rapamycin or a derivative thereof is a concentration of about 450 nM. In some embodiments, the effective amount of rapamycin or a derivative thereof is a concentration of about 500 nM. In some embodiments, the effective amount of rapamycin or a derivative thereof is a concentration of about 550 nM. In some embodiments, the effective amount of rapamycin or a derivative thereof is a concentration of about 600 nM. In some embodiments, the effective amount of rapamycin or a derivative thereof is a concentration of about 650 nM. In some embodiments, the effective amount of rapamycin or a derivative thereof is a concentration of about 700 nM. In some embodiments, the effective amount of rapamycin or a derivative thereof is a concentration of about 750 nM. In some embodiments, the effective amount of rapamycin or a derivative thereof is a concentration of about 800 nM. In some embodiments, the effective amount of rapamycin or a derivative thereof is a concentration of about 850 nM. In some embodiments, the effective amount of rapamycin or a derivative thereof is a concentration of about 900 nM. In some embodiments, the effective amount of rapamycin or a derivative thereof is a concentration of about 1000 nM. In some embodiments, the effective amount of rapamycin or a derivative thereof is a concentration greater than 1000 nM.

In specific embodiments, provided herein is a method for generating mitochondria replaced lymphoid cells, wherein the method comprises incubating lymphoid cells that have not undergone a procedure to reduce or deplete endogenous mitochondria with isolated exogenous mitochondria and about 100 nM to about 1000 nM of rapamycin for a sufficient period of time to non-invasively transfer the exogenous mitochondria to the lymphoid cells, thereby generating a mitochondria replaced lymphoid cells.

In one embodiment, the lymphoid cells that have not undergone a procedure to reduce or deplete endogenous mitochondria are incubated with isolated exogenous mitochondria and about 100 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 100 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 100 nM of an mTOR inhibitor for about 36 hours. In another embodiment, the lymphoid cells that have not undergone a procedure to reduce or deplete endogenous mitochondria are incubated with isolated exogenous mitochondria and about 100 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 100 nM of an mTOR inhibitor for about 2 days. In another embodiment, the lymphoid cells that have not undergone a procedure to reduce or deplete endogenous mitochondria are incubated with isolated exogenous mitochondria and about 100 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 100 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 200 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 200 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 200 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 200 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 200 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 200 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 200 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 300 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 300 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 300 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 300 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 300 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 300 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 300 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 400 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 400 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 400 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 400 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 400 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 400 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 400 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 500 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 500 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 500 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 500 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 500 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 500 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 500 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 600 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 600 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 600 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 600 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 600 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 600 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 600 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 700 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 700 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 700 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 700 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 700 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 700 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 700 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 800 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 800 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 800 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 800 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 800 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 800 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 800 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 900 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 900 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 900 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 900 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 900 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 900 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 900 nM of an mTOR inhibitor (such as an mTOR inhibitor described in Section 6.2, including, e.g., rapamycin) for more than 7 days.

In another embodiment, the lymphoid cells that have not undergone a procedure to reduce or deplete endogenous mitochondria are incubated with isolated exogenous mitochondria and about 1000 nM of an mTOR inhibitor (such as an mTOR inhibitor described in Section 6.2, including, e.g., rapamycin) for about 12 hours. In another embodiment, the lymphoid cells that have not undergone a procedure to reduce or deplete endogenous mitochondria are incubated with isolated exogenous mitochondria and about 1000 nM of an mTOR inhibitor (such as an mTOR inhibitor described in Section 6.2, including, e.g., rapamycin) for about 24 hours. In another embodiment, the lymphoid cells that have not undergone a procedure to reduce or deplete endogenous mitochondria are incubated with isolated exogenous mitochondria and about 1000 nM of an mTOR inhibitor (such as an mTOR inhibitor described in Section 6.2, including, e.g., rapamycin) for about 36 hours. In another embodiment, the lymphoid cells that have not undergone a procedure to reduce or deplete endogenous mitochondria are incubated with isolated exogenous mitochondria and about 1000 nM of an mTOR inhibitor (such as an mTOR inhibitor described in Section 6.2, including, e.g., rapamycin) for about 48 hours. In another embodiment, the lymphoid cells that have not undergone a procedure to reduce or deplete endogenous mitochondria are incubated with isolated exogenous mitochondria and about 1000 nM of an mTOR inhibitor (such as an mTOR inhibitor described in Section 6.2, including, e.g., rapamycin) for about 2 days. In another embodiment, the lymphoid cells that have not undergone a procedure to reduce or deplete endogenous mitochondria are incubated with isolated exogenous mitochondria and about 1000 nM of an mTOR inhibitor (such as an mTOR inhibitor described in Section 6.2, including, e.g., rapamycin) for about 2 to 7 days. In another embodiment, the lymphoid cells that have not undergone a procedure to reduce or deplete endogenous mitochondria are incubated with isolated exogenous mitochondria and about 1000 nM of an mTOR inhibitor (such as an mTOR inhibitor described in Section 6.2, including, e.g., rapamycin) for more than 7 days.

In some embodiments, the mTOR inhibitor (such as an mTOR inhibitor described in Section 6.2 including, e.g., rapamycin) is washed out and/or diluted in the cell culture after incubation with the lymphoid cells and exogenous mitochondria for a certain period of time. For example, in some embodiments, the lymphoid cells are incubated with exogenous mitochondria and the mTOR inhibitor (such as an mTOR inhibitor described in Section 6.2 including, e.g., rapamycin) for about 12 hours or more before the culture media is exchanged for fresh media without the mTOR inhibitor, and then the cells are cultured at least another 12 hours. In some embodiments, the lymphoid cells are incubated with exogenous mitochondria and the mTOR inhibitor (such as an mTOR inhibitor described in Section 6.2 including, e.g., rapamycin) for about 24 hours or more before the culture media is exchanged. In some embodiments, the lymphoid cells are incubated with exogenous mitochondria and the mTOR inhibitor (such as an mTOR inhibitor described in Section 6.2 including, e.g., rapamycin) for about 36 hours or more before the culture media is exchanged. In some embodiments, the lymphoid cells are incubated with exogenous mitochondria and the mTOR inhibitor (such as an mTOR inhibitor described in Section 6.2 including, e.g., rapamycin) for more than 48 hours before the culture media is exchanged. In some embodiments, the lymphoid cells are incubated with exogenous mitochondria and the mTOR inhibitor for about 60 hours or more before the culture media is exchanged. In some embodiments, the lymphoid cells are rinsed one or more times with a suitable buffer that maintains the lymphoid cell's water balance (e.g., PBS, Hank's balanced salt solution (HBSS), Earle's balanced salt solution (EBSS), culture medium—with or without supplements, etc.) prior to culturing in fresh culture media that does not contain the mTOR inhibitor. In some embodiments, the lymphoid cells are incubated with the mTOR inhibitor and exogenous mitochondria and the media is not exchanged for the duration of the culture.

In some embodiments, the mTOR inhibitor (such as an mTOR inhibitor described in Section 6.2, including, e.g., rapamycin) is diluted from the cell culture after incubation with the lymphoid cells and exogenous mitochondria. For example, in some embodiments, the lymphoid cells are incubated with exogenous mitochondria and the mTOR inhibitor (such as an mTOR inhibitor described in Section 6.2, including, e.g., rapamycin) for about 12 hours or more before a portion of the culture media is exchanged for fresh media (e.g., about 25%, about 50%, or about 75% of the culture media is exchanged for fresh media) without the mTOR inhibitor such that the concentration of the mTOR inhibitor is diluted, and then the cells are cultured at least another 12 hours (e.g., about 12 hours, about 18 hours, about 24 hours, about 36 hours, about 48 hours, about 72 hours, about 96 hours, about 120 hours, 132 hours, about 148 hours, about 172 hours, about 200 hours, about 250 hours, about 275 hours or more). In some embodiments, the lymphoid cells are incubated with exogenous mitochondria and the mTOR inhibitor (such as an mTOR inhibitor described in Section 6.2, including, e.g., rapamycin) for about 24 hours or more before a portion of the culture media is exchanged for fresh media (e.g., about 25%, about 50%, or about 75% of the culture media is exchanged for fresh media) without the mTOR inhibitor such that the concentration of the mTOR inhibitor is diluted, and then the cells are cultured at least another 12 hours (e.g., about 12 hours, about 18 hours, about 24 hours, about 36 hours, about 48 hours, about 72 hours, about 96 hours, about 120 hours, 132 hours, about 148 hours, about 172 hours, about 200 hours, about 250 hours, about 275 hours or more). In some embodiments, the lymphoid cells are incubated with exogenous mitochondria and the mTOR inhibitor (such as an mTOR inhibitor described in Section 6.2, including, e.g., rapamycin) for 36 hours or more before a portion of the culture media is exchanged for fresh media (e.g., about 25%, about 50%, or about 75% of the culture media is exchanged for fresh media) without the mTOR inhibitor such that the concentration of the mTOR inhibitor is diluted, and then the cells are cultured at least another 12 hours (e.g., about 12 hours, about 18 hours, about 24 hours, about 36 hours, about 48 hours, about 72 hours, about 96 hours, about 120 hours, 132 hours, about 148 hours, about 172 hours, about 200 hours, about 250 hours, about 275 hours or more). In some embodiments, the lymphoid cells are incubated with exogenous mitochondria and the mTOR inhibitor for 48 hours or more before a portion of the culture media is exchanged for fresh media (e.g., about 25%, about 50%, or about 75% of the culture media is exchanged for fresh media) without the mTOR inhibitor such that the concentration of the mTOR inhibitor is diluted, and then the cells are cultured at least another 12 hours (e.g., about 12 hours, about 18 hours, about 24 hours, about 36 hours, about 48 hours, about 72 hours, about 96 hours, about 120 hours, 132 hours, about 148 hours, about 172 hours, about 200 hours, about 250 hours, about 275 hours or more). In some embodiments, the lymphoid cells are incubated with exogenous mitochondria and the mTOR inhibitor (such as an mTOR inhibitor described in Section 6.2, including, e.g., rapamycin) for 60 hours or more before a portion of the culture media is exchanged for fresh media (e.g., about 25%, about 50%, or about 75% of the culture media is exchanged for fresh media) without the mTOR inhibitor such that the concentration of the mTOR inhibitor is diluted, and then the cells are cultured at least another 12 hours (e.g., about 12 hours, about 18 hours, about 24 hours, about 36 hours, about 48 hours, about 72 hours, about 96 hours, about 120 hours, 132 hours, about 148 hours, about 172 hours, about 200 hours, about 250 hours, about 275 hours or more). In some embodiments, the lymphoid cells are incubated with exogenous mitochondria and the mTOR inhibitor (such as an mTOR inhibitor described in Section 6.2, including, e.g., rapamycin) and the media is not exchanged for the duration of the culture.

The mitochondria replaced lymphoid cells that are generated according to the methods provided herein can include various types of lymphoid cells. Non-limiting examples of lymphoid cells suitable for use with the present disclosure include T cells, B cells, monocytes, macrophages, natural killer (NK) cells, or granulocytes.

In some embodiments, the lymphoid cells are T cells. In certain embodiments, the T cells are CD4+ T cells. In some embodiments, the T cells are CD8+ T cell. In certain embodiments, the lymphoid cells comprise a combination of CD4+ T cells and CD8+ T cells. In some embodiments, the lymphoid cells are or comprise Tregs. In certain embodiments, the lymphoid cells are or comprise effector T cells. In some embodiments, the lymphoid cells are or comprise memory T cells, effector T cells, Tregs, or a combination thereof. In specific embodiments, the lymphoid cells are or comprise T cells that include an exogenous polynucleotide encoding a T cell receptor (TCR) or a chimeric antigen receptor (CAR). In specific embodiments, the lymphoid cells are or comprise T cells that have been genetically modified to express a T cell receptor (TCR) or a chimeric antigen receptor (CAR).

In some embodiments, the T cells are T cells genetically modified to express a chimeric antigen receptor (CAR) or a T cell receptor (TCR). For example, TCRs use naturally occurring receptors that can also recognize antigens that are inside tumor cells. CARs, on the other hand, comprise portions of an antibody that can recognize a specific antigen only on the surface of cancer cells. Despite their use in immunotherapy CAR-T cells and TCR T cells can become exhausted. Thus, as provided herein, in some embodiments, the mitochondria replaced lymphoid cells produced according to the method described here, such as the methods described in Section 6.1, can be CAR-T cells or TCR T cells, and be administered to a subject to treat or ameliorate a symptom of a cancer. Non-limiting exemplary types of cancer that can benefit from the mitochondria replaced lymphoid cells described herein that are or comprise CAR-T cells or TCR T include blood cancers (e.g., acute lymphatic leukemia, multiple myeloma, B cell lymphoma, mantel cell lymphoma), as well as solid tumors. In specific embodiments, the subject is a human subject.

CARs are generally designed to include an extracellular target-binding domain, a hinge region, a transmembrane domain that anchors the CAR to the cell membrane, and one or more intracellular domains that transmit activation signals. Depending on the number of costimulatory domains, CARs can be classified into first generation (an intracellular domain, e.g., CD3ζ, only), second generation (one costimulatory domain and an intracellular domain), or third generation CARs (more than one costimulatory domain and an intracellular domain). New generation CARs are also being developed (see, e.g., Guedan S, et al. Mol Ther Methods Clin Dev. 2018 Dec. 31; 12:145-156). CAR targets for hematological malignancies (e.g., CD19, BCMA) and CAR targets for solid tumors (e.g., HER2, PSCA) are known in the art, and any target is suitable for use with the present disclosure (see, e.g., Dotti G, et al. Immunol Rev. 2014; 257(1): 107-126). CAR-T cells can be autologous or allogeneic to the subject receiving administration of the CAR-T cells. In some embodiments, the CAR-T cells are allogeneic, relative to the subject. In other embodiments, the CAR-T cells are autologous, relative to the subject.

In certain embodiments, the CAR comprises a tumor antigen recognition domain, a transmembrane domain, and one or more intracellular signaling domains. In some embodiments, the CAR comprises a tumor antigen recognition domain, a transmembrane domain, one or more costimulatory molecules, and one or more intracellular signaling domains. In a specific embodiment, the CAR comprises a tumor antigen recognition domain, a transmembrane domain, and an intracellular domain. In a specific embodiment, the CAR comprises a tumor antigen recognition domain, a transmembrane domain, an intracellular domain and at least one costimulatory domain. In another specific embodiment, the CAR comprises a tumor antigen recognition domain, a transmembrane domain, two or more costimulatory domains and an intracellular domain. In some embodiments, the CAR comprises a constitutively or inducibly expressed chemokine. In certain embodiments, the CAR comprises intracellular domains of a cytokine receptor (e.g. IL-2Rβ chain fragment).

In some embodiments, the T cells are T cells genetically modified to express a TCR. TCRs generally use heterodimers consisting of alpha and beta peptide chains to recognize polypeptide fragments presented by MHC molecules, and the TCR T cells are genetically engineered TCR products that can recognize specific antigens. Generally, the artificially designed high-affinity TCR is encoded in T cells by genetic engineering technology, which enhances both specificity recognition and affinity during the recognition of tumor cells by T cells. TCR-T cells can be autologous or allogeneic to the subject receiving administration of the TCR-T cells. In some embodiments, the TCR-T cells are allogeneic, relative to the subject. In other embodiments, the TCR-T cells are autologous, relative to the subject.

In certain embodiments, the TCR T cells recognize an antigen on a hematological malignancy (e.g., CMV, WT1, HA-1). In certain embodiments, the TCR T cells recognize an antigen on a solid tumors (e.g., HBV, p53, mutant KRAS). In certain embodiments, the TCR T cells recognize SL9 associated with HIV.

In some embodiments, the lymphoid cells that are subject to a method described herein are dysfunctional T cells. For example, in some embodiments, the T cells comprise exhausted T cells, senescent T cells, or a combination thereof. In certain embodiments, the lymphoid cells comprise T cells isolated from a subject (e.g., a human subject) with a disease or disorder associated with inherited and acquired mitochondrial DNA mutations, such as a mitochondrial disease, or an immunological deficiency associated with heteroplasmic immune cells.

The present method for generating mitochondria replaced lymphoid cells can involve human and non-human cells. In some embodiments, the lymphoid cells are human lymphoid cells. In other embodiments, the lymphoid cells are non-human lymphoid cells (e.g., murine lymphoid cells, monkey lymphoid cells, etc.).

In certain embodiments, the lymphoid cells subject to a method described herein are isolated from a human subject with a with a disease or disorder associated with inherited and acquired mitochondrial DNA mutations, such as a mitochondrial disease, or an immunological deficiency associated with heteroplasmic immune cells. In some embodiments, the lymphoid cells subject to a method described herein are isolated from the subject (e.g., human subject) to be administered mitochondria replaced lymphoid cells as a therapy. In other words, the mitochondria replaced lymphoid cells are derived from lymphoid cells autologous the subject to be treated with the mitochondria replaced lymphoid cells. In other embodiments, the lymphoid cells subject to a method described herein are isolated from a different subject (e.g., a different human subject) than the subject (e.g., human subject) to be administered mitochondria replaced lymphoid cells as a therapy. In other words, in specific embodiments, the mitochondria replaced lymphoid cells are derived from lymphoid cells allogenic the subject to be treated with the mitochondria replaced lymphoid cells. In some embodiments, the lymphoid cells are T cells. In certain embodiments, the lymphoid cells are CD4+ T cells, CD8+ T cells, or a combination thereof. In some embodiments, the lymphoid cells are T cells genetically modified to express a CAR.

Techniques known to one of skill in the art or described herein (e.g., described in the Example) may be used to isolate lymphoid cells from a subject (e.g., human subject). For example, peripheral blood lymphocytes may be isolated from a subject (e.g., a human subject). In some embodiments, a certain subset of lymphoid cells (e.g., a subset of T cells, such as CD4+ T cells, CD8+ T cells or a combination thereof) may be isolated from the peripheral blood lymphocytes using techniques known to one of skill in the art or described herein, such as magnetic isolation.

The amount of the isolated exogenous mitochondria that is incubated with lymphoid cells to generate mitochondria replaced lymphoid cells will depend on factors, such as the amount of lymphoid cells are being co-incubated with the isolated mitochondria. Generally, the amount of isolated exogenous mitochondria incubated with lymphoid cells is about 5 μg to about 100 μg per 1×10⁶ lymphoid cells. In some embodiments, the amount of isolated exogenous mitochondria incubated with lymphoid cells is about 10 μg to about 90 μg per 1×10⁶ lymphoid cells. In some embodiments, the amount of isolated exogenous mitochondria incubated with lymphoid cells is about 20 μg to about 80 μg per 1×10⁶ lymphoid cells. In some embodiments, the amount of isolated exogenous mitochondria incubated with lymphoid cells is about 30 μg to about 70 μg per 1×10⁶ lymphoid cells. In some embodiments, the amount of isolated exogenous mitochondria incubated with lymphoid cells is about 40 μg to about 80 μg per 1×10⁶ lymphoid cells. In some embodiments, the amount of isolated exogenous mitochondria incubated with lymphoid cells is about 5 μg per 1×10⁶ lymphoid cells. In some embodiments, the amount of isolated exogenous mitochondria incubated with lymphoid cells is about 10 μg per 1×10⁶ lymphoid cells. In some embodiments, the amount of isolated exogenous mitochondria incubated with lymphoid cells is about 20 μg per 1×10⁶ lymphoid cells. In some embodiments, the amount of isolated exogenous mitochondria incubated with lymphoid cells is about 30 μg per 1×10⁶ lymphoid cells. In some embodiments, the amount of isolated exogenous mitochondria incubated with lymphoid cells is about 40 μg per 1×10⁶ lymphoid cells. In some embodiments, the amount of isolated exogenous mitochondria incubated with lymphoid cells is about 50 μg per 1×10⁶ lymphoid cells. In some embodiments, the amount of isolated exogenous mitochondria incubated with lymphoid cells is about 60 μg per 1×10⁶ lymphoid cells. In some embodiments, the amount of isolated exogenous mitochondria incubated with lymphoid cells is about 70 μg per 1×10⁶ lymphoid cells. In some embodiments, the amount of isolated exogenous mitochondria incubated with lymphoid cells is about 80 μg per 1×10⁶ lymphoid cells. In some embodiments, the amount of isolated exogenous mitochondria incubated with lymphoid cells is about 90 μg per 1×10⁶ lymphoid cells. In some embodiments, the amount of isolated exogenous mitochondria incubated with lymphoid cells is about 100 μg per 1×10⁶ lymphoid cells. In some embodiments, the amount of isolated exogenous mitochondria incubated with lymphoid cells is greater than 100 μg per 1×10⁶ lymphoid cells.

As provided herein, the isolated exogenous mitochondria of the present disclosure can be obtained from various types of cells that have a healthy and functional mitochondria. Assays for determining mitochondrial function are known in the art, and include assays such as those described in Section 6.4. Exemplary sources of mitochondria for use in the methods provided herein include fibroblasts, platelet cells, as well as other lymphoid cells. In certain embodiments, the isolated exogenous mitochondria are obtained from a fibroblast. In some embodiments, the isolated exogenous mitochondria are obtained from a platelet cell. In some embodiments, the isolated exogenous mitochondria are obtained from a lymphoid cell.

As provided herein, the isolated exogenous mitochondria can be autologous or allogeneic to the recipient cell. In some embodiments, the isolated exogenous mitochondria is allogeneic, relative to the recipient cell. For example, the isolated exogenous mitochondria can be obtained from a subject that is different from the recipient cell. In other embodiments, the isolated exogenous mitochondria is autologous. By way of example, an exemplary autologous isolated exogenous mitochondria can include mitochondria isolated from the same subject at an earlier point in time, such as from the placenta or umbilical cord blood. Another exemplary autologous exogenous mtDNA can include, for example, donor mtDNA that has been isolated from the same subject as the recipient cell and modified prior to replacing it with the recipient cell.

In certain embodiments, mitochondria are obtained from normal peripheral blood collected using a leukopak (i.e., an enriched leukapheresis product). In some embodiments, the mitochondria are obtained from CD34+ cells.

Mitochondrial isolation may be accomplished by any of a number of well-known techniques including, but not limited to those described herein. In certain embodiments, the exogenous mitochondria for use in mitochondrial transfer is isolated using a commercial kit, such as, for example, the Qproteum mitochondria isolation kit (Qiagen, USA), or the MITOISO2 mitochondria isolation kit (Sigma, USA). In other embodiments, the exogenous mitochondria for use in mitochondrial transfer is isolated manually (see, e.g., Preble et al. J. Vis. Exp. 2014, 91: e51682; Gasnier et al. Anal Biochem 1993; 212(1):173-8 and Frezza et al. Nat Protoc 2007; 2(2): 287-95). For example, an exemplary manual isolation of mitochondria includes isolating the mitochondria from donor cells by pelleting the donor cells, washing the cell pellet of 1-2 mL derived from approximately 10⁹ cells grown in culture, swelling the cells in a hypotonic buffer, rupturing the cells with a Dounce or Potter-Elvehjem homogenizer using a tight-fitting pestle, and isolating the mitochondria by differential centrifugation. Manual isolation can also include, for example, sucrose density gradient ultracentrifugation, or free-flow electrophoresis. Without wishing to be bound by any particular method, it is understood that the kits and manual methods described herein are exemplary, and that any mitochondrial isolation method can be used, and would be within the skill set of a person skilled in the art.

In some embodiments, the isolated donor mitochondria is substantially pure of other organelles. In other embodiments, the isolated mitochondria can contain impurities and is enriched for mitochondria. For example, in some embodiments, the isolated mitochondria are about 90% pure, about 80% pure, about 70% pure, about 60%, pure, about 50%, pure, or any integer in-between. In general, it is understood that any impurities contained with the isolated donor mitochondria will not affect the viability or function of the recipient cell upon mitochondrial transfer. In specific embodiments, the transfer of the exogenous mitochondria, exogenous mtDNA, or a combination thereof does not involve transfer of non-mitochondrial organelles.

The quantity and quality of isolated mitochondria can easily be determined by a number of well-known techniques including but not limited to those described herein, and in the cited references. For example, in some embodiments, the quantity of isolated mitochondria is determined by assessment of total protein content. Various methods are available for measurement of total protein content, such as the Biuret and Lowry procedures (see, e.g., Hartwig et al., Proteomics, 2009 June; 9(11):3209-14), as well as the Bradford protein assay (Bradford. Anal Biochem. 1976; 72:248-54). In other embodiments, the quantity of isolated mitochondria is determined by mtDNA copy number.

The period of time sufficient to non-invasively transfer the exogenous mitochondria to the lymphoid cells can be any period of time that results in a detectable amount of exogenous mtDNA that is greater than lymphoid cells not subjected to exogenous mitochondria. In some embodiments, the period of time sufficient to non-invasively transfer the exogenous mitochondria to the lymphoid cells is any period of time that results in at least 20% of exogenous mtDNA being transferred to the lymphoid cells relative to the amount of exogenous mtDNA transferred to lymphoid cells not incubated with the exogenous mtDNA. In certain embodiments, the period of time sufficient to non-invasively transfer the exogenous mitochondria to the lymphoid cells is any period of time that results in at least 30% of exogenous mtDNA being transferred to the lymphoid cells relative to the amount of exogenous mtDNA transferred to lymphoid cells not incubated with the exogenous mtDNA. In some embodiments, the period of time sufficient to non-invasively transfer the exogenous mitochondria to the lymphoid cells is any period of time that results in at least 30% of exogenous mtDNA being transferred to the lymphoid cells relative to the amount of exogenous mtDNA transferred to lymphoid cells not incubated with the exogenous mtDNA. In certain embodiments, the period of time sufficient to non-invasively transfer the exogenous mitochondria to the lymphoid cells is any period of time that results in at least 40% of exogenous mtDNA being transferred to the lymphoid cells relative to the amount of exogenous mtDNA transferred to lymphoid cells not incubated with the exogenous mtDNA. In some embodiments, the period of time sufficient to non-invasively transfer the exogenous mitochondria to the lymphoid cells is any period of time that results in at least 50% of exogenous mtDNA being transferred to the lymphoid cells relative to the amount of exogenous mtDNA transferred to lymphoid cells not incubated with the exogenous mtDNA. In some embodiments, the period of time sufficient to non-invasively transfer the exogenous mitochondria to the lymphoid cells is any period of time that results in at least 60% of exogenous mtDNA being transferred to the lymphoid cells relative to the amount of exogenous mtDNA transferred to lymphoid cells not incubated with the exogenous mtDNA. In some embodiments, the period of time sufficient to non-invasively transfer the exogenous mitochondria to the lymphoid cells is any period of time that results in at least 70% of exogenous mtDNA being transferred to the lymphoid cells relative to the amount of exogenous mtDNA transferred to lymphoid cells not incubated with the exogenous mtDNA. Techniques known to one of skill in the art or described herein (e.g., in the Example) may be used to assess the transfer of exogenous mtDNA to lymphoid cells. Generally, the sufficient period of time is at least approximately 12 hours and less than two weeks. In some embodiments, the sufficient period of time is at least 12 hours. In some embodiments, the sufficient period of time is at least 24 hours. In some embodiments, the sufficient period of time is at least 36 hours. In some embodiments, the sufficient period of time is at least 48 hours. In some embodiments, the sufficient period of time is approximately 2 days or more. In some embodiments, the sufficient period of time is approximately 7 days or more. In some embodiments, the sufficient period of time is approximately 2 days to approximately 7 days.

In some embodiments, the ratio of the copy number of exogenous mtDNA to the copy number of endogenous mtDNA in the mitochondria replaced lymphoid cell generated according to the methods provided herein is greater than 4 to 1. In some embodiments, the ratio is about 4 to 1. In some embodiments, the ratio is about 3 to 1. In some embodiments, the ratio is about 2 to 1. In some embodiments, the ratio is about 1 to 1. In some embodiments, the ratio is about 0.75 to 1. In some embodiments, the ratio is about 0.5 to 1. In some embodiments, the ratio is about 0.25 to 1. In some embodiments, the ratio is about 0.1 to 1.

In general, the methods provided herein are compatible with simple co-incubation of the lymphoid cell and the isolated exogenous mitochondria. However, it is also possible to promote the transfer of mitochondria by optionally centrifuging the lymphoid cells and the isolated exogenous mitochondria. In some embodiments, the lymphoid cells have been treated with an mTOR inhibitor (such as an mTOR inhibitor described in Section 6.2, including, e.g., rapamycin) prior to centrifuging the lymphoid cells and isolated exogenous mitochondria. In some embodiments, the mTOR inhibitor, the lymphoid cells and isolated exogenous mitochondria are centrifuged together prior to incubation together as described herein. In other embodiments, the mTOR inhibitor is added after centrifuging the lymphoid cells and isolated exogenous mitochondria.

In another aspect, provided herein is a method for generating mitochondria replaced lymphoid cells, the method comprising: (a) centrifuging lymphoid cells and isolated exogenous mitochondria under conditions sufficient to generate a cell pellet, wherein the lymphoid cells have not undergone a procedure to reduce or deplete endogenous mitochondria; and (b) incubating the lymphoid cells (e.g., T cells or other lymphoid cells as described herein) with 100 nM to 1000 nM of rapamycin for approximately 24 hours or more, thereby generating mitochondria replaced lymphoid cells.

Incubation of the lymphoid cells following centrifuging with rapamycin can be for any time sufficient to generate mitochondria replaced lymphoid cells. In one embodiment, incubating is for approximately 7 days or more. In one embodiment, incubating is for approximately 2 days to approximately 7 days. In one embodiment, the medium is changed with 50% fresh medium during in the incubation period.

Centrifugation conditions can readily by determined by a person skilled in the art, and the speed and time can vary so long as the cell and mitochondria are not damaged, and transfer of the mitochondria is promoted. By way of example, centrifuging conditions can include centrifuging at approximately 1,500 relative centrifugal force (RCF, also termed “g”) for approximately 5 minutes at room temperature. In a specific embodiment, centrifuging is as described in the Example, infra.

In one embodiment, centrifuging is at approximately 500 RCF for approximately 5 minutes at room temperature. In another embodiment, centrifuging is at approximately 750 RCF for approximately 5 minutes at room temperature. In another embodiment, centrifuging is at approximately 1,000 RCF for approximately 5 minutes at room temperature. In another embodiment, centrifuging is at approximately 1,500 RCF for approximately 5 minutes at room temperature. In another embodiment, centrifuging is at approximately 2,000 RCF for approximately 5 minutes at room temperature. In another embodiment, centrifuging is at approximately 2,500 RCF for approximately 5 minutes at room temperature. In another embodiment, centrifuging is at approximately 3,000 RCF for approximately 5 minutes at room temperature.

In one embodiment, centrifuging is at approximately 500 RCF for approximately 10 minutes at room temperature. In another embodiment, centrifuging is at approximately 750 RCF for approximately 10 minutes at room temperature. In another embodiment, centrifuging is at approximately 1,000 RCF for approximately 10 minutes at room temperature. In another embodiment, centrifuging is at approximately 1,500 RCF for approximately 10 minutes at room temperature. In another embodiment, centrifuging is at approximately 2,000 RCF for approximately 10 minutes at room temperature. In another embodiment, centrifuging is at approximately 2,500 RCF for approximately 10 minutes at room temperature. In another embodiment, centrifuging is at approximately 3,000 RCF for approximately 10 minutes at room temperature.

In another embodiment, centrifuging is at approximately 500 RCF for approximately 15 minutes at room temperature. In another embodiment, centrifuging is at approximately 750 RCF for approximately 15 minutes at room temperature. In another embodiment, centrifuging is at approximately 1,000 RCF for approximately 15 minutes at room temperature. In another embodiment, centrifuging is at approximately 1,500 RCF for approximately 15 minutes at room temperature. In another embodiment, centrifuging is at approximately 2,000 RCF for approximately 15 minutes at room temperature. In another embodiment, centrifuging is at approximately 2,500 RCF for approximately 15 minutes at room temperature. In another embodiment, centrifuging is at approximately 3,000 RCF for approximately 15 minutes at room temperature.

In another embodiment, centrifuging is at approximately 500 RCF for less than an hour at room temperature. In another embodiment, centrifuging is at approximately 750 RCF for less than an hour at room temperature. In another embodiment, centrifuging is at approximately 1,000 RCF for less than an hour at room temperature. In another embodiment, centrifuging is at approximately 1,500 RCF for less than an hour at room temperature. In another embodiment, centrifuging is at approximately 2,000 RCF for less than an hour at room temperature. In another embodiment, centrifuging is at approximately 2,500 RCF for less than an hour at room temperature. In another embodiment, centrifuging is at approximately 3,000 RCF for less than an hour at room temperature.

In one embodiment, centrifuging is at approximately 500 RCF for approximately 5 minutes at about 4° C. In another embodiment, centrifuging is at approximately 750 RCF for approximately 5 minutes at about 4° C. In another embodiment, centrifuging is at approximately 1,000 RCF for approximately 5 minutes at about 4° C. In another embodiment, centrifuging is at approximately 1,500 RCF for approximately 5 minutes at about 4° C. In another embodiment, centrifuging is at approximately 2,000 RCF for approximately 5 minutes at about 4° C. In another embodiment, centrifuging is at approximately 2,500 RCF for approximately 5 minutes at about 4ºC. In another embodiment, centrifuging is at approximately 3,000 RCF for approximately 5 minutes at about 4° C.

In another embodiment, centrifuging is at approximately 500 RCF for approximately 10 minutes at about 4° C. In another embodiment, centrifuging is at approximately 750 RCF for approximately 10 minutes at about 4° C. In another embodiment, centrifuging is at approximately 1,000 RCF for approximately 10 minutes at about 4° C. In another embodiment, centrifuging is at approximately 1,500 RCF for approximately 10 minutes at about 4° C. In another embodiment, centrifuging is at approximately 2,000 RCF for approximately 10 minutes at about 4° C. In another embodiment, centrifuging is at approximately 2,500 RCF for approximately 10 minutes at about 4° C. In another embodiment, centrifuging is at approximately 3,000 RCF for approximately 10 minutes at about 4° C.

In another embodiment, centrifuging is at approximately 500 RCF for approximately 15 minutes at about 4° C. In another embodiment, centrifuging is at approximately 750 RCF for approximately 15 minutes at about 4° C. In another embodiment, centrifuging is at approximately 1,000 RCF for approximately 15 minutes at about 4° ° C. In another embodiment, centrifuging is at approximately 1,500 RCF for approximately 15 minutes at about 4° C. In another embodiment, centrifuging is at approximately 2,000 RCF for approximately 15 minutes at about 4° C. In another embodiment, centrifuging is at approximately 2,500 RCF for approximately 15 minutes at about 4° C. In another embodiment, centrifuging is at approximately 3,000 RCF for approximately 15 minutes at about 4° C.

In another embodiment, centrifuging is at approximately 500 RCF for less than an hour at about 4° C. In another embodiment, centrifuging is at approximately 750 RCF for less than an hour at about 4° C. In another embodiment, centrifuging is at approximately 1,000 RCF for less than an hour at about 4° C. In another embodiment, centrifuging is at approximately 1,500 RCF for less than an hour at about 4° C. In another embodiment, centrifuging is at approximately 2,000 RCF for less than an hour at about 4° C. In another embodiment, centrifuging is at approximately 2,500 RCF for less than an hour at about 4° C. In another embodiment, centrifuging is at approximately 3,000 RCF for less than an hour at about 4° C. In some embodiments, the methods provided herein do not involve centrifugation.

In certain embodiments, lymphoid cells are starved for a certain period of time before incubating the lymphoid cells with an mTOR inhibitor (such as an mTOR inhibitor described in Section 6.2, including, e.g., rapamycin) and isolated exogenous mitochondria. For example, in some embodiments, the lymphoid cells are starved for 3-6 hours, 3-9 hours, 6-9 hours, 6-12 hours, 6-18 hours, 12-24 hours or 24-48 hours prior to incubating the lymphoid cells with an mTOR inhibitor (such as an mTOR inhibitor described in Section 6.2, including, e.g., rapamycin) and isolated exogenous mitochondria. In certain embodiments, the lymphoid cells are starved by depriving the lymphoid cells of glucose, an essential amino acid (e.g., glutamine), and/or serum. For example, in some embodiments, the lymphoid cells are cultured in cell culture media that lacks one or more nutrients (e.g., glucose-free, serum-free, and/or glutamine-free).

In certain embodiments, lymphoid cells are starved for a certain period of time before centrifuging the lymphoid cells with isolated exogenous mitochondria. For example, in some embodiments, the lymphoid cells are starved for 3-6 hours, 3-9 hours, 6-9 hours, 6-12 hours, 6-18 hours, 12-24 hours, or 24-48 hours prior to centrifuging the lymphoid cells with isolated exogenous mitochondria. In certain embodiments, the lymphoid cells are starved by depriving the lymphoid cells of glucose, an essential amino acid (e.g., glutamine), and/or serum. After the centrifugation, the lymphoid cells (e.g., T cells or other lymphoid cells as described herein) may be incubated with an effective amount of an mTOR inhibitor (such as an mTOR inhibitor described in Section 6.2, including, e.g., rapamycin) for a sufficient period of time to non-invasively transfer the exogenous mitochondria to the lymphoid cells, as described above. In some embodiments, the lymphoid cells are cultured in cell culture media that lacks one or more nutrients (e.g., glucose-free, serum-free, and/or glutamine-free) prior to centrifuging the lymphoid cells with isolated exogenous mitochondria.

In certain embodiments, incubating the lymphoid cells with an mTOR inhibitor (such as an mTOR inhibitor described in Section 6.2, including, e.g., rapamycin) and isolated exogenous mitochondria is performed under starvation conditions. In certain embodiments, the lymphoid cells are starved by depriving the lymphoid cells of glucose, an essential amino acid (e.g., glutamine), and/or serum. For example, in some embodiments, lymphoid cells are incubated in cell culture that (1) lacks one or more nutrients (e.g., glucose-free, serum-free, and/or glutamine-free) and (2) contains an effective amount of an mTOR inhibitor (such as an mTOR inhibitor described in Section 6.2, including, e.g., rapamycin). In some embodiments, incubating the lymphoid cells with an mTOR inhibitor (such as an mTOR inhibitor described in Section 6.2, including, e.g., rapamycin) and isolated exogenous mitochondria is performed under conditions that does not involve starvation.

In a specific embodiment, provided herein is a method for generating mitochondria replaced lymphoid cells using the method described in Example 8.1.

In specific embodiments, mitochondria replaced lymphoid cells exhibit one, two or more improvements in function relative to the lymphoid cells from which the mitochondria replaced lymphoid cells are generated. See, e.g., Section 6.4 for functions of lymphoid cells that may be improved in mitochondria replaced lymphoid cells.

6.2 Rapamycin Analogs and Other mTOR Inhibitors

Various compounds are known to inhibit mTOR, including rapamycin, also known as sirolimus (CAS Number 53123-88-9; C51H79NO13), and rapamycin derivatives (e.g., rapamycin analogs, also known as “rapalogs”). Non-limiting examples of rapamycin derivatives include, for example, temsirolimus (CAS Number 162635-04-3; C56H87NO16), everolimus (CAS Number 159351-69-6; C53H83NO14), ridaforolimus (CAS Number 572924-54-0; C53H84NO14P), WYE-125132 (WYE-132), and Zotarolimus (ABT-578). In a specific embodiment, the mTOR inhibitor is rapamycin.

In some embodiments, the mTOR inhibitor suitable for use in any of the methods described herein inhibits both mTORC1 and mTORC2, such as, for example AZD8055, Torin 1, Torkinib or Omipalisib.

In certain embodiments, the mTOR inhibitor disclosed herein also inhibits one or more substrates other than mTOR, such as a dual kinase inhibitor. Inhibitors with specificity to mTOR and one or more substrates are known in the art. By way of example, dual PI3K/mTOR inhibitors are one type of mTOR inhibitor suitable for use with the present disclosure that inhibit mTOR and another substrate. Non-limiting examples of dual PI3K/mTOR inhibitors include, for example, Dactolisib (also known as BEZ235), PI-103, Bimiralisib (also known as PQR309), GDC-0084, and Gedatolisib.

### 6.3 Methods of Treatment

As provided herein, the mitochondria replaced lymphoid cells generated according to the methods of the present disclosure are suitable for use as a cell-based therapies, such as in the methods described in Section 6.3.1 and Section 6.3.2. For example, in some embodiments, an effective amount of the mitochondria replaced lymphoid cells generated according to the methods described in Section 6.1, can be combined with a pharmaceutically acceptable carrier to result in a pharmaceutical composition. In some embodiments, provided herein is a composition (e.g., a pharmaceutical composition) comprising mitochondria replaced lymphoid cells generated according to the methods described herein, such as in the methods described in Section 6.1, and a pharmaceutically acceptable carrier. In some embodiments, provided herein is a composition (e.g., a pharmaceutical composition) comprising an effective amount of mitochondria replaced lymphoid cells generated according to the methods described herein, such as in the methods described in Section 6.1, and a pharmaceutically acceptable carrier.

As used herein, the term “pharmaceutically acceptable” when used in reference to a carrier, is intended to mean that the carrier, diluent or excipient is not toxic or otherwise undesirable, (i.e., the material may be administered to a subject without causing any undesirable biological effects), and it is compatible with the other ingredients of the formulation. The term “carrier” refers to a diluent, adjuvant, excipient, or vehicle with which the therapeutic is administered. Such pharmaceutical carriers can be sterile liquids, such as saline solutions in water. A saline solution can be a carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents.

In certain embodiments, the effective amount of the mitochondria replaced lymphoid cells is about 1×10⁶ to about 1×10⁷ cells. In some embodiments, the effective amount of the mitochondria replaced lymphoid cells is about 10×10⁶ to about 900×10⁶ cells. In some embodiments, the effective amount of the mitochondria replaced lymphoid cells is about 50×10⁶ to about 800×10⁶ cells. In some embodiments, the effective amount of the mitochondria replaced lymphoid cells is about 100×10⁶ to about 700×10⁶ cells. In some embodiments, the effective amount of the mitochondria replaced lymphoid cells is about 200×10⁶ to about 900×10⁶ cells. In some embodiments, the effective amount of the mitochondria replaced lymphoid cells is about 250×10⁶ to 750×10⁶ cells. In some embodiments, the effective amount of the mitochondria replaced lymphoid cells is about 50×10⁶ cells. In some embodiments, the effective amount of the mitochondria replaced lymphoid cells is about 150×10⁶ cells. In some embodiments, the effective amount of the mitochondria replaced lymphoid cells is about 300×10⁶ cells. In some embodiments, the effective amount of the mitochondria replaced lymphoid cells is about 450×10⁶ cells. In some embodiments, the effective amount of the mitochondria replaced lymphoid cells is about 600×10⁶ cells. In some embodiments, the effective amount of the mitochondria replaced lymphoid cells is about 850×10⁶ cells.

In particular embodiments, the effective amount of the effective amount of the mitochondria replaced lymphoid cells is determined empirically, such as, for example, based on the weight of the subject, or the burden of the disease or disorder. In some embodiments, the effective amount of the mitochondria replaced lymphoid cells is about 1.0×10⁶ cells/kg to about 1.0×10⁷ cells/kg. In some embodiments, the effective amount of the mitochondria replaced lymphoid cells is about 1.0×10⁶ cells/kg to about 500×10⁶ cells/kg. In some embodiments, the effective amount of the mitochondria replaced lymphoid cells is about 1.0×10⁶ cells/kg to about 50×10⁶ cells/kg. In some embodiments, the effective amount of the mitochondria replaced lymphoid cells is about 1.0×10⁶ cells/kg to about 10×10⁶ cells/kg. In some embodiments, the effective amount of the mitochondria replaced lymphoid cells is about 1.0×10⁶ cells/kg to about 5.0×10⁶ cells/kg. In some embodiments, the effective amount of the mitochondria replaced lymphoid cells is about 10×10⁶ cells/kg to about 600×10⁶ cells/kg. In some embodiments, the effective amount of the mitochondria replaced lymphoid cells is about 50×10⁶ cells/kg to about 750×10⁶ cells/kg. In some embodiments, the effective amount of the mitochondria replaced lymphoid cells is about 1.0×10⁶ cells/kg. In some embodiments, the effective amount of the mitochondria replaced lymphoid cells is about 2.5×10⁶ cells/kg. In some embodiments, the effective amount of the mitochondria replaced lymphoid cells is about 5.0×10⁶ cells/kg. In some embodiments, the effective amount of the mitochondria replaced lymphoid cells is about 10.0×10⁶ cells/kg. In some embodiments, the effective amount of the mitochondria replaced lymphoid cells is about 50.0×10⁶ cells/kg. In some embodiments, the effective amount of the mitochondria replaced lymphoid cells is about 250.0×10⁶ cells/kg. In some embodiments, the effective amount of the mitochondria replaced lymphoid cells is about 500×10⁶ cells/kg.

In certain embodiments, treatment with mitochondria replaced lymphoid cells results one, two, or more, or all of the following: (1) a reduction in severity, progression, spread, and/or frequency of one or more symptoms, (2) elimination of one or more symptoms and/or underlying cause, (3) prevention of the occurrence of one or more symptoms and/or their underlying cause, and (4) improvement or remediation of damage. In specific embodiments, treatment includes therapeutic treatment as well as prophylactic, or suppressive measures for the condition, disease or disorder.

In some embodiments, the mitochondria replaced lymphoid cells for use as a cell-based therapies, such as in the methods described in Section 6.3.1 and Section 6.3.2, are autologous or allogeneic to the subject receiving administration of the mitochondria replaced lymphoid cells. In some embodiments, the mitochondria replaced lymphoid cells for use as a cell-based therapies, such as in the methods described in Section 6.3.1 and Section 6.3.2, are autologous to the subject receiving administration of the mitochondria replaced lymphoid cells. In some embodiments, the mitochondria replaced lymphoid cells for use as a cell-based therapies, such as in the methods described in Section 6.3.1 and Section 6.3.2, are allogeneic to the subject receiving administration of the mitochondria replaced lymphoid cells.

### 6.3.1 Methods of Treating a Mitochondrial Disease or Disorder

In one aspect, provided herein is a method for ameliorating a symptom of mitochondrial complex III deficiency to a subject in need thereof, comprising administering to the subject an effective amount of the mitochondria replaced lymphoid cells generated according to the methods described in Section 6.1, and a pharmaceutically acceptable carrier.

Mitochondrial complex III is essential for regulatory T cell (Treg) suppressive function. For example, it has been shown that Treg cells require mitochondrial complex III to maintain immune regulatory gene expression and suppressive function (see Weinberg, S. et al. Nature vol. 565,7740 (2019): 495-499). Mitochondrial complex III deficiency is a genetic condition. It is generally caused by mutations in nuclear DNA in the BCSIL, UQCRB and UQCRQ genes and inherited in an autosomal recessive manner. However, it may also be caused by mutations in mitochondrial DNA in the MTCYB gene, which is passed down maternally or occurs sporadically and may result in a milder form of the condition.

Accordingly, in some embodiments, the mitochondria replaced lymphoid cells are Treg cells and the mitochondria replaced lymphoid cells are administered to a subject having mitochondrial complex III deficiency. In specific embodiments, the subject is a human subject.

In certain embodiments, the method for ameliorating a symptom of mitochondrial complex III deficiency includes a reduction in severity, progression, spread, and/or frequency of symptoms, elimination of symptoms and/or underlying cause, prevention of the occurrence of symptoms and/or their underlying cause, and improvement or remediation of damage. In one embodiment, the method for ameliorating a symptom of mitochondrial complex III deficiency includes a reduction the severity of the symptom. In one embodiment, the method for ameliorating a symptom of mitochondrial complex III deficiency includes a reduction in the progression of the symptom. In another embodiment, the method for ameliorating a symptom of mitochondrial complex III deficiency includes a reduction in the spread of the symptom. In another embodiment, the method for ameliorating a symptom of mitochondrial complex III deficiency reduces the frequency of the symptom. In another embodiment, the method for ameliorating a symptom of mitochondrial complex III deficiency includes an elimination of the symptom. In another embodiment, the method for ameliorating a symptom of mitochondrial complex III deficiency comprises prevention of the occurrence of symptoms of the mitochondrial complex III deficiency. In another embodiment, the method for ameliorating a symptom of mitochondrial complex III deficiency comprises improvement of damage from the mitochondrial complex III deficiency. In another embodiment, the method for ameliorating a symptom of mitochondrial complex III deficiency comprises a remediation of damage from the mitochondrial complex III deficiency.

6.3.2 Methods of Treating a Immunological Deficiency Associated with Heteroplasmic Immune Cells.

Also provided herein is a method for treating an immunological deficiency associated with heteroplasmic immune cells to a subject in need thereof, comprising administering to the subject an effective amount of the mitochondria replaced lymphoid cells generated according to the methods described in Section 6.1, and a pharmaceutically acceptable carrier.

In certain embodiments, the heteroplasmic immune cells are a result of unwanted pharmacological side effects. For example, nucleoside reverse transcriptase inhibitors (NRTIs) inhibit human immunodeficiency virus (HIV)-1 replication, and are useful in the treatment of HIV. However, NRTIs also exhibit side effects in human tissues that appear to result from NRTI inhibition of human mitochondrial polymerase γ (pol γ). In one embodiment, the subject has received a reverse transcriptase inhibitor. In one embodiment, the subject has human immunodeficiency virus (HIV). In specific embodiments, the subject is human.

Similarly, many of the drugs approved by the FDA for the treatment of hepatitis B virus (HBV) target the reverse transcriptase (RT or P gene product) and are nucleoside RT inhibitors (NRTIs) that suppress viral replication. Thus, in some embodiments, the subject has hepatitis B virus (HBV). In specific embodiments, the subject is human.

Heteroplasmy can also arise from various types of mutations. Accordingly, the methods provided herein as not limited to treating heteroplasmic immune cells caused by a NRTI inhibitors.

In some embodiments, treating an immunological deficiency associated with heteroplasmic immune cells includes a reduction in severity, progression, spread, and/or frequency of symptoms, elimination of symptoms and/or underlying cause, prevention of the occurrence of symptoms and/or their underlying cause, and improvement or remediation of damage. In one embodiment, treating an immunological deficiency associated with heteroplasmic immune cells comprises a reduction in severity of the immunological deficiency. In another embodiment, treating an immunological deficiency associated with heteroplasmic immune cells comprises a reduction in progression of the immunological deficiency. In another embodiment, treating an immunological deficiency associated with heteroplasmic immune cells comprises a reduction in spread of the immunological deficiency. In another embodiment, treating an immunological deficiency associated with heteroplasmic immune cells comprises a reduction in frequency of symptoms of the immunological deficiency. In another embodiment, treating an immunological deficiency associated with heteroplasmic immune cells comprises an elimination of the immunological deficiency. In another embodiment, treating an immunological deficiency associated with heteroplasmic immune cells comprises prevention of the occurrence of symptoms of the immunological deficiency. In another embodiment, treating an immunological deficiency associated with heteroplasmic immune cells comprises improvement of damage from the immunological deficiency. In another embodiment, treating an immunological deficiency associated with heteroplasmic immune cells comprises a remediation of damage from the immunological deficiency.

### 6.4 Biological Assays for Lymphoid Function

Successful generation of mitochondria replaced lymphoid cells results in lymphoid cells that have improved function, relative to lymphoid cells without mitochondria replacement. Various functional assays can be used to measure and evaluate phenotype of the mitochondria replaced lymphoid cells.

In some embodiments, the mitochondria replaced lymphoid cells have improved mitochondrial function, relative to lymphoid cells without mitochondria replacement. A person skilled in the art would understand how to evaluate mitochondrial function. For example, cell-based assays, such as the Seahorse Bioscience XF Extracellular Flux Analyzer, can used performed for the determination of basal oxygen consumption, glycolysis rates, ATP production, and respiratory capacity to assess mitochondrial dysfunction. Similarly, the Oroboros 02K respirometer can also be used to establish quantitative functional mitochondrial diagnosis. It is understood that the assay examples described above are exemplary and are not inclusive of all methods to evaluate mitochondrial function.

Increased cell proliferation can also be an indicator of improved lymphocyte function. An exemplary assay for measuring cell proliferation of lymphocytes is a mixed lymphocyte reaction (MLR) assay. The MLR assay generally involves combining a population of the mitochondria replaced lymphoid cells, such as CD4+ T cells, with a different population of lymphocytes and measuring proliferation. In some embodiments, the mitochondria replaced lymphoid cells generated according to the methods provided herein have increased cell proliferation, as compared to a lymphoid cell that has not been incubated with isolated exogenous mitochondria and an mTOR inhibitor.

Another exemplary assay that can be used to assess the function of the mitochondria replaced cells, such as in cytotoxic T cells, is a cytotoxic T cell (CTL) assay. A CTL assay indicates the presence and cytotoxic activity of T cells to a specific antigen and allows to examine the influence of a test item on this immune function. Accordingly, in some embodiments, the mitochondria replaced lymphoid cells generated according to the methods provided herein have increased CTL response, as compared to a lymphoid cell that has not been incubated with isolated exogenous mitochondria and an mTOR inhibitor.

DNA stability is important for the function of T cells. Therefore, another non-limiting exemplary assay that can be used to assess the function of the mitochondria replaced cells is measuring the DNA damage response. Double-strand breaks (DSBs) are critical damage to genome stability, thus are quickly and precisely repaired to maintain cellular homeostasis. An early response to DSBs is the phosphorylation of the minor histone H2A variant at the position of Ser-139, which form γH2AX. Thus, by way of example, DNA damage response can be assayed by detecting phosphorylation of histone 2A X (H2AX), and phosphorylation can be measured using any assay known in the art, such as for example by flow cytometry or by immunoblot.

For example, the following assay may be conducted to detect DNA damage response: lymphoid cells (e.g., T cells) are washed with PBS twice, and suspended and chilled with 70% ethanol at −20° C. for 60 minutes, then washed with PBS twice, and stained with PE anti-H2A.X phospho antibody (Biolegend) and APC mouse anti-CD3 antibody (Biolegend) at 37° C. in a humidified 5% CO2 incubator for 60 minutes. Cells are then washed and re-suspended with autoMACS™ Running Buffer (Miltenyi Biotec, Bergisch Gladbach, Germany), and immediately followed by flow cytometry analysis. Data may be analyzed using FlowJo software (BD Bioscience, Franklin Lakes, NJ, USA).

Ca2+ signaling is critical to lymphoid cell (e.g., T cell) activation as a means of rapidly activating and integrating numerous signaling pathways to generate widespread changes in gene expression and function. Various assays to measure Ca2+ signaling are known in the art (See Samakai E, et al., Signaling Mechanisms Regulating T Cell Diversity and Function. Boca Raton (FL): CRC Press/Taylor & Francis; 2018. Chapter 10.) Thus, in some embodiments, the mitochondria replaced lymphoid cells generated according to the methods provided herein have increased Ca2+ signaling, as compared to a lymphoid cell that has not been incubated with isolated exogenous mitochondria and an mTOR inhibitor.

Telomere length can also serve as an indicator of mitochondria replaced cell's function. Telomere length can be measured using any method known in the art. One exemplary technique is by measuring absolute telomere length by qPCR. Thus, in some embodiments, the mitochondria replaced lymphoid cells generated according to the methods provided herein have a decreased telomere shortening, as compared to a lymphoid cell that has not been incubated with isolated exogenous mitochondria and an mTOR inhibitor.

As provided herein, in some embodiments the lymphoid cells that are used to generate a mitochondria replaced cell are senescent and the mitochondria replaced cell exhibits a decrease in senescence. Thus, measure of Senescence Associated Secretory Phenotype (SASP) can serve as a functional assay. SASP includes increased secretion of inflammatory cytokines (e.g., interferon gamma (IFNγ) and/or tumor necrosis factor alpha (TNFα), growth factors, and proteases, as well as reduced and/or slower rates of cell population doublings, shortened telomeres, increased DNA damage response (DDR), or a combination thereof. FACS analysis for senescence markers (e.g., CD57/KIR/KLRG1) can also be employed. Accordingly, in some embodiments, the mitochondria replaced lymphoid cells generated according to the methods provided herein have a decreased in senescence, as compared to a lymphoid cell that has not been incubated with isolated exogenous mitochondria and an mTOR inhibitor.

In some embodiments the lymphoid cells that are used to generate a mitochondria replaced cell are exhausted T cells and the mitochondria replaced cell exhibits a reduction in T cell exhaustion. FACS analysis for exhaustion markers (e.g., PD-1/TIM3/LAG3) can be used to measure T cell exhaustion. Accordingly, in some embodiments, the mitochondria replaced lymphoid cells generated according to the methods provided herein have a reduction in exhaustion, as compared to a lymphoid cell that has not been incubated with isolated exogenous mitochondria and an mTOR inhibitor.

### 7. EMBODIMENTS

This invention provides the following non-limiting embodiments.

A1. A method for generating mitochondria replaced lymphoid cells in which at least 20% of endogenous mitochondrial DNA (mtDNA) has been replaced with exogenous mtDNA, wherein the method comprises incubating lymphoid cells that have not undergone a procedure to reduce or deplete endogenous mitochondria with isolated exogenous mitochondria and an effective amount of mammalian target of rapamycin (mTOR) inhibitor for a sufficient period of time to non-invasively transfer the exogenous mitochondria to the lymphoid cells, thereby generating mitochondria replaced lymphoid cells in which at least 20% of the endogenous mtDNA has been replaced with exogenous mtDNA.

A2. The method of claim A1, wherein the mTOR inhibitor comprises rapamycin or a derivative thereof.

A3. The method of embodiment A1 or embodiment A2, wherein the mTOR inhibitor is rapamycin.

A4. The method of any one of embodiments A1 to A3, wherein the effective amount of the mTOR inhibitor is a concentration of about 100 nM to about 1000 nM.

A5. The method of any one of embodiments A1 to A3, wherein the effective amount of the mTOR inhibitor is a concentration of about 200 nM to about 500 nM.

A6. The method of any one of embodiments A1 to A3, wherein the effective amount of the mTOR inhibitor is a concentration of about 100 nM.

A7. The method of any one of embodiments A1 to A3, wherein the effective amount of the mTOR inhibitor is a concentration of about 200 nM.

A8. The method of any one of embodiments A1 to A3, wherein the effective amount of the mTOR inhibitor is a concentration of about 500 nM.

A9. The method of any one of embodiments A1 to A3, wherein the effective amount of the mTOR inhibitor is a concentration of about 1000 nM

A10. The method of any one of embodiments A1 to A9, wherein the mitochondria replaced lymphoid cells comprises at least 20% of exogenous mtDNA and no more than 80% endogenous mtDNA, as measured by TaqMan Single Nucleotide Polymorphism (SNP) Assay.

A11. A method for generating mitochondria replaced lymphoid cells, wherein the method comprises incubating lymphoid cells that have not undergone a procedure to reduce or deplete endogenous mitochondria with isolated exogenous mitochondria and about 100 nM to about 1000 nM of rapamycin for a sufficient period of time to non-invasively transfer the exogenous mitochondria to the lymphoid cells, thereby generating a mitochondria replaced lymphoid cells.

A12. The method of any one of embodiments A1 to A11, wherein the isolated exogenous mitochondria is about 20 μg to 80 μg protein per 1×10⁶ cells.

A13. The method of any one of embodiments A1 to A12, further comprising centrifuging the lymphoid cells prior to incubating.

A14. The method of embodiment A13, wherein centrifuging is performed at 1,500 relative centrifugal force (RCF) for approximately 5 minutes at room temperature.

A15. The method of any one of embodiments A1 to A14, wherein the sufficient period of time is at least approximately 24 hours.

A16. The method of any one of embodiments A1 to A14, wherein the sufficient period of time is at least 36 hours.

A17. The method of any one of embodiments A1 to A14, wherein the sufficient period of time is at least 48 hours.

A18. The method of any one of embodiments A1 to A14, wherein the sufficient period of time is approximately 2 days or more.

A19. The method of any one of embodiments A1 to A14, wherein the sufficient period of time is approximately 7 days or more.

A20. The method of any one of embodiments A1 to A14, wherein the sufficient period of time is approximately 2 days to approximately 7 days.

A21. A method for generating mitochondria replaced lymphoid cells, wherein the method comprises:
(a) centrifuging lymphoid cells and isolated exogenous mitochondria under conditions sufficient to generate a cell pellet, wherein the lymphoid cells have not undergone a procedure to reduce or deplete endogenous mitochondria; and
(b) incubating the lymphoid cells with 100 nM to 1000 nM of rapamycin for approximately 24 hours or more, thereby generating mitochondria replaced lymphoid cells.

A22. The method of embodiment A21, wherein incubating is for approximately 7 days or more.

A23. The method of embodiment A21, wherein incubating is for approximately 2 days to approximately 7 days.

A24. The method of any one of embodiments A1 to A23, wherein the lymphoid cells are T cells, B cells, monocytes, macrophages, natural killer (NK) cells, or granulocytes.

A25. The method of embodiment A24, wherein the lymphoid cells are T cells.

A26. The method of embodiment A25, wherein the T cells comprise exhausted T cells, senescent T cells, or a combination thereof.

A27. The method of any one of embodiments A1 to A26, wherein the lymphoid cells are human lymphoid cells.

A28. A composition comprising an effective amount of the mitochondria replaced lymphoid cells generated by the method of any one of embodiments A1 to A27, and a pharmaceutically acceptable carrier.

A29. A method for ameliorating a symptom of mitochondrial complex III deficiency to a subject in need thereof, comprising administering to the subject the composition of embodiment A28.

A30. A method for treating an immunological deficiency associated with heteroplasmic immune cells to a subject in need thereof, comprising administering to the subject the composition of embodiment A28.

A31. The method of embodiment A30, wherein the subject has received a reverse transcriptase inhibitor.

A32. The method of embodiment A31, wherein the subject has human immunodeficiency virus (HIV).

A33. The method of embodiment A31, wherein the subject has hepatitis B virus (HBV).

A34. The method of any one of embodiments A29 to A33, wherein the subject is human.

### 8. EXAMPLE

The examples in this section are offered by way of illustration, and not by way of limitation. The following examples are presented as exemplary embodiments of the invention.

They should not be construed as limiting the broad scope of the invention.

### 8.1 Example 1: Methods and Compositions for Treating Mitochondria Diseases and Other Diseases Associated with Heteroplasmy with Lymphoid Cells

This example demonstrates that endogenous mitochondria and/or mtDNA may be replaced in lymphocytes (e.g., T lymphocytes) by exogenous mitochondria using rapamycin without prior depletion of the endogenous mtDNA.

### 8.1.1 Materials & Methods

Isolation and Cell Culture of Primary Mouse T lymphocytes: All the animal experiments were performed according to the animal experiment guidelines issued by the Animal Care and Use Committee of our institution (M2019-536). For mouse T lymphocytes isolation, spleens were removed from mouse after anesthetized and sacrificed. Spleens were washed with PBS (FUJIFILM Wako Pure Chemical Corp.), then mashed and filtrated to extract splenocytes. Mouse T cells were highly purified from splenocytes by immunomagnetic negative selection using EasySep mouse T cell isolation kit (Veritas, Santa Clara, CA, USA) according to the manufacturer's recommendations. Isolated mouse T cells were cultured in Advanced RPMI1640 medium supplemented with 10% FBS, 1% penicillin/streptomycin, 20 mM L-glutamine (Thermo Fisher Scientific incorporated), 20 μM recombinant human IL-2 (PeproTech, Rocky Hill, NJ, USA), which is activated with Dynabeads mouse T-activator CD3/CD28 (Thermo Fisher Scientific incorporated). Cells were incubated at 37ºC in a humidified 5% CO2 incubator.

Mitochondrial Isolation and Transfer to Mouse T cells: Mitochondria were isolated from B6 murine embryonic fibroblasts (MEF). In brief, the cells were harvested from culture dishes with homogenization buffer [HB; 20 mM HEPES-KOH (pH 7.4), 220 mM mannitol and 70 mM sucrose] containing a protease inhibitor mixture (Sigma-Aldrich, St. Louis, Missouri, USA). The cell pellet was resuspended in HB and incubated on ice for 5 min. The cells were ruptured by 10 strokes of a 27-gauge needle on ice. The homogenate was centrifuged (400×g, 4° C.; 5 min.) two times to remove unbroken cells. The mitochondria were harvested by centrifugation (6000×g, 4° C.; 5 min.) and resuspended in HB. The amounts of isolated mitochondria were expressed as protein concentration using a Bio-Rad protein assay kit (Bio-Rad Laboratories, incorporated, Richmond, CA, USA). The isolated mitochondria are mixed with mouse T cells in standard medium, and centrifuged at 1,500 g for 5 minutes at room temperature. The pellet is gently resuspended and incubated with adding various concentration of rapamycin at 37° C.′ under 5% CO2 for 24 h.

Isolation and Cell Culture of Human T cells: Venous heparinized blood was taken according to the standard procedures from the median cubital vein. Human peripheral blood mononuclear cells (PBMC) were isolated from human peripheral blood using density-gradient centrifugation with 1.077 g/ml percoll (GE Healthcare Life Sciences, Buckinghamshire, England). Cells were cultured in TexMACS medium (Miltenyi Biotec) supplemented with 10% fetal bovine serum, 1% penicillin/streptomycin (Thermo Fisher Scientific incorporated), 20 μM of IL-7, and 10 μM of IL-15 on cell culture plates coated with anti-CD3 antibody and anti-CD28 antibody (Miltenyi Biotec). Cells were incubated at 37° C. in a humidified 5% CO2 incubator.

Mitochondrial Isolation and Transfer to Human T cells: The immortalized human uterine endometrial gland-derived mesenchymal cell line, EPC100 (Japanese Collection of Research Bioresources Cell Bank, JCRB1538) was used as an exemplary donor of exogenous mitochondria for mitochondrial transfer to human primary T cells by using the same protocol as that of murine protocol.

MtDNA sequencing: To purify the mtDNAs from genomic DNAs (Jayaprakash A D. Nucleic Acids Res. 2015), we removed only nuclear DNA using Exonuclease V (ExoV, New England Biolabs Ltd, Ipswich, MA, USA). Briefly, the whole DNAs were extracted from cells using NucleoSpin Tissue (MACHEREY-NAGEL) and heated at 70° C. for 30 min to inactivate any left-over proteinase K. At first digestion, the whole DNA samples (4-8 μg in 35 μl) were added in the following steps, 10× New England Biolabs (NEB)4 Buffer (6 μl), 10 mM ATP (12 μl), ExoV from NEB-M0345S (4 μl) and H₂O (3 μl). The digests were left at 37ºC for 48 h, heat-inactivated at 70° ° C. for 30 min and purified using N Wizard SV Gel and PCR Clean-Up System (Promega Corporation, Madison, WI, USA). At second digestion, the ExoV treated DNAs (in 35 μl) were added in the following, NEB4 10× Buffer (6 μl), 10 mM ATP (12 μl), ExoV from NEB-M0345S (4 μl) and H₂O (3 μl). The digests were left at 37ºC for 16 h, heat-inactivated at 70° ° C. for 30 min and purified using Wizard SV Gel and PCR Clean-Up System (Promega Corporation, Madison, WI, USA). These ExoV treated mtDNAs were processed for deep sequencing. The sequencing data is generated as fastq format files. The unmapped reads were filtered for quality (sequences with >10 consecutive nucleotides with Q<20 were eliminated) and mapped to the reference mitochondrial genome (GRCh38), and converted to BAM files. These files were analyzed for heteroplasmy with mtDNA-server (mtdna-server.uibk.ac.at/index.html).

TaqMan Single Nucleotide Polymorphism (SNP) Assay: Based upon the differences in the mitochondrial HVR1 sequences of human GJ T cells and EPC100 cells, and the mitochondrial ND1 of B6 mouse and NZB mouse, the probes and primer set forth in Tables 1 and 2 were designed, which provide sensitivity and specificity in the TaqMan SNP assay. To determine mutation ratios, we designed wild-type and mutant allele-specific TaqMan probes for the TaqMan SNP assay. The extracted DNA (1 ng) was used for quantitative PCR with the TaqMan Universal PCR Master Mix kit (Thermo Fisher Scientific Incorporated) on a CFX connect real-time system (Bio-Rad Laboratories, Incorporated) under the following conditions: 40 cycles of PCR (95° ° C. for 15 see and 60° ° C. for 1 min) after initial denaturation (95° ° C. for 10 min). A calibration curve was created using known CNs of plasmids containing the amplified mtDNA fragments for the targets. The mtDNA copy number was estimated from the content ratio of 12S rRNA on mtDNA and ACTB (or Actb) on nuclear DNA by delta cycle threshold-based relative quantification.

TABLE 1

| | Fluorescent | |
|---|---|---|
| | Dye | Sequence (5′ to 3′) |
| Human T cell- | FAM | CAAcCAACCcTCAAC |
| probe | | (SEQ ID NO: 1) |
| EPC100-probe | VIC | AGCAAtCAACCtTCAAC |
| | | (SEQ ID NO: 2) |
| NZB mouse | FAM | TGTTCTTTATTAATgcCCTAAC |
| | | (SEQ ID NO: 3) |
| B6 mouse | VIC | TCTTATTAATatCCTAACACTCC |
| | | (SEQ ID NO: 4) |

TABLE 2

| | Sequence (5′ to 3′) |
|---|---|
| Human HVR1-F primer | CCCCATGCTTACAAGCAAGTAC- |
| | (SEQ ID NO: 5) |
| Human HVR1-R primer | TTGGAGTTGCAGTTGATGTGTG |
| | (SEQ ID NO: 6) |
| Mouse ND1-F primer | CCTTGTTCCCAGAGGTTCAAATC |
| | (SEQ ID NO: 7) |
| Mouse ND1-R primer | TGCCGTATGGACCAACAATG |
| | (SEQ ID NO: 8) |

### 8.1.2 Results

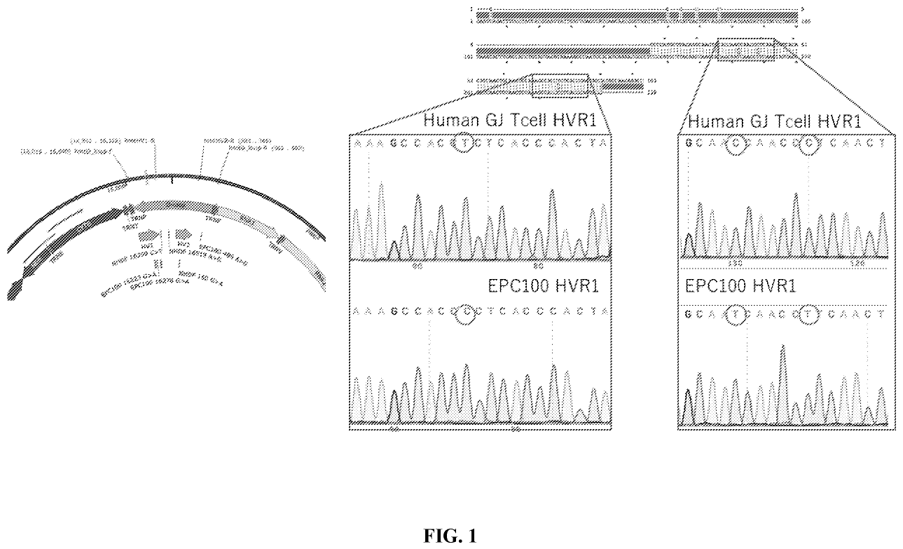

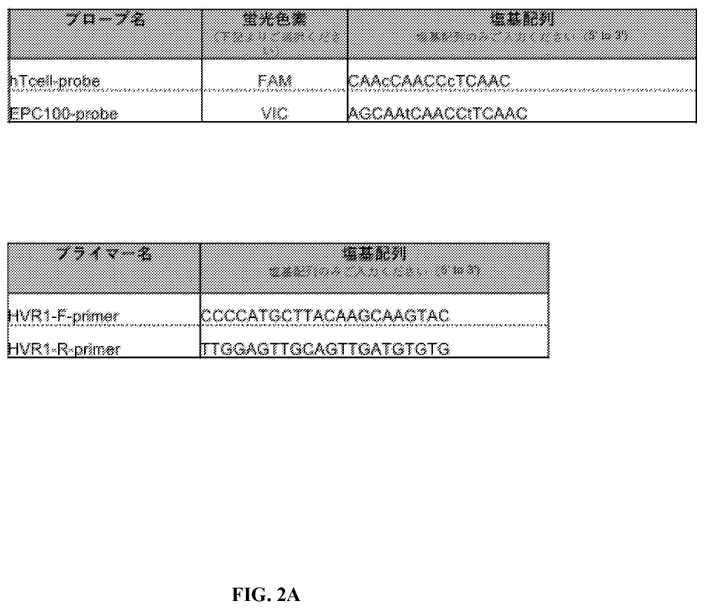

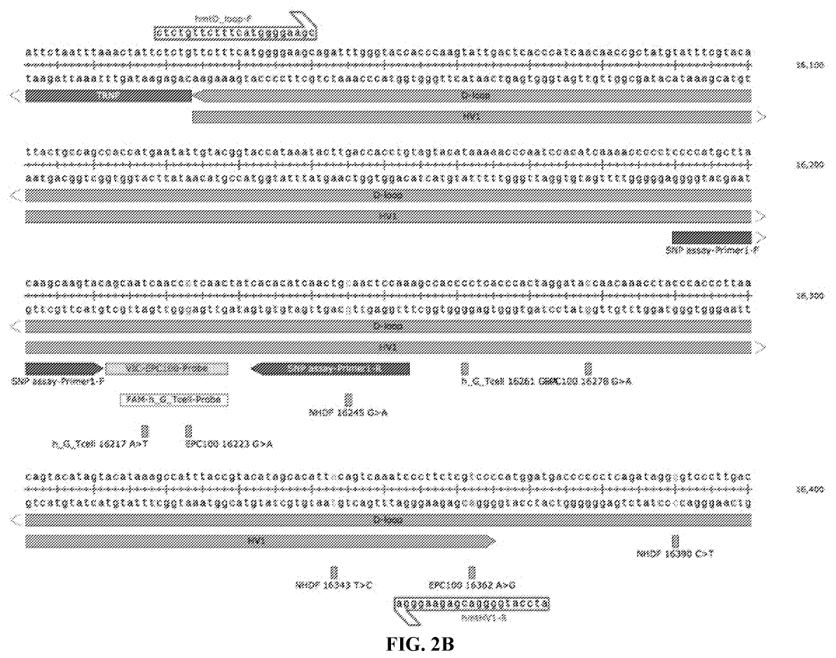

As shown in FIG. 1, differences in mtDNA sequences from human GJ T cells and EPC100 cells were detected by sequencing the D Loop, and some differences were observed in the D Loop hypervariable region (“HVR”). Based upon the differences in the D Loop HVR, a set of probes and primers were designed (FIG. 2A) for use in an SNP assay to detect mitochondria replacement. The assay was validated by using a cell population at a given mixing rate of human GJ T cells and EPC100 cells as a test sample prior to the experimental sample. The assay demonstrated the accurate ratio of genotype according to the mixing rate of the cell population (data not shown). Accordingly, the TaqMan probes can readily detect and discriminate between mtDNA from GJ T cells and mtDNA from EPC100 cells. Thus, the SNP assay is a useful tool for being able to identify and discriminate between mtDNA from the two different sources.

Human GJ T cells were cultured in vitro for 2 days in TexMacs with 5% FBS and IL-7 and IL-15. Mitochondria transfer was performed by culturing the human GJ T cells in the absence of rapamycin or presence of varying concentrations of rapamycin (50 nM, 100 nM, 200 nM, or 500 nM) with or without mitochondria from EPC100 cells (“Day 0”). 24 hours after culturing in the presence of rapamycin, 50% of the media was replenished with TexMacs with 5% FBS and IL-7 and IL-15, but no additional rapamycin was added. Following the media replacement, the cells were cultured for either one or six additional days, for a total of two or seven days total post-mitochondria transfer.

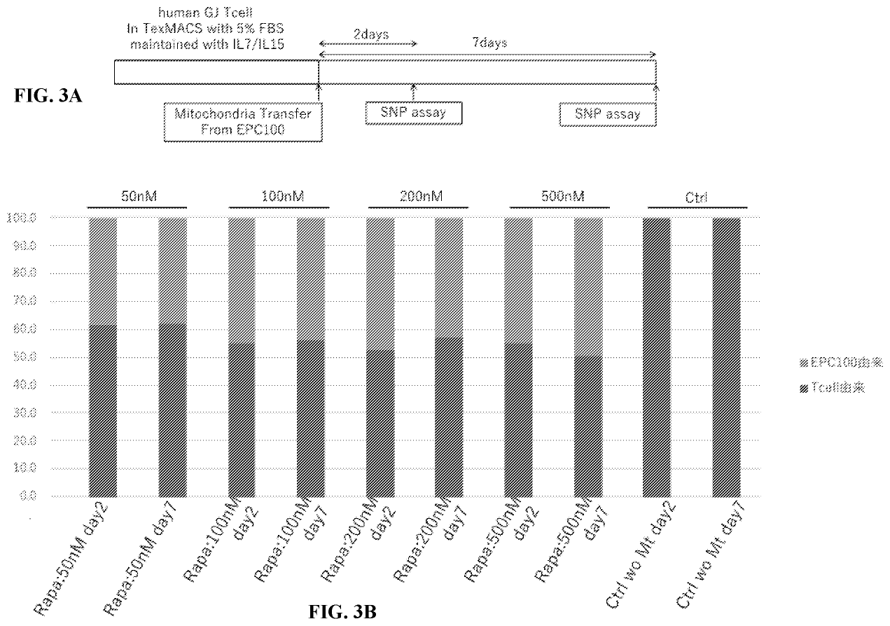

Two days and seven days after culturing the human GJ T cells with the mitochondria from EPC 100 cells, SNP assays were conducted to detect the transfer of mitochondria from EPC 100 cells to human GJ T cells. See FIG. 3A for the outline of the protocol. As shown in FIG. 3B, mtDNA from EPC100 cells was detected in human GJ T cells cultured in the presence of 50 to 500 nM of rapamycin for 2 days and 7 days, whereas no mtDNA from EPC100 cells was detected in human GJ T cells cultured without EPC100 mitochondria. The mtDNA content in T cells that were contacted with donor mitochondria in the presence of rapamycin consisted of between 40-50% of mtDNA from EPC100 cells, with the amount of donor mtDNA increasing with increasing amount of rapamycin. For example, the portion of donor mtDNA detected in T cells after contact with donor mitochondria in the presence of 50 nM rapamycin was less than 40% after 7 days, whereas the portion of donor mtDNA detected in T cells after contact with donor mitochondria in the presence of 500 nM rapamycin was approximately 50% after 7 days. Thus, no mitochondrial depletion was required to achieve a successful transfer of mtDNA to T cells when the T cells are cultured in rapamycin.

3×10⁶ mouse NZB T cells per well were cultured in Advanced RPMI with 10% FBS and IL-2/CD3/CD28 with 40 μg mitochondria from B6 MEF in the absence of rapamycin or the presence of varying concentrations of rapamycin (100 nM, 200 nM, 500 nM or 1000 NM) without depletion of the NZB T cell mtDNA. B6 T cells, which was used as the mitochondrial donor, have 91 polymorphism in its mtDNA compared with mtDNA from the current inbred laboratory mice including B6 T cells. 24 hours after incubation with rapamycin, 50% of the media was replenished with Advanced RPMI with 10% FBS and IL-2/CD3/CD28, but no additional rapamycin was added. Cells were cultured for a total of two or seven days before evaluating mitochondrial replacement.

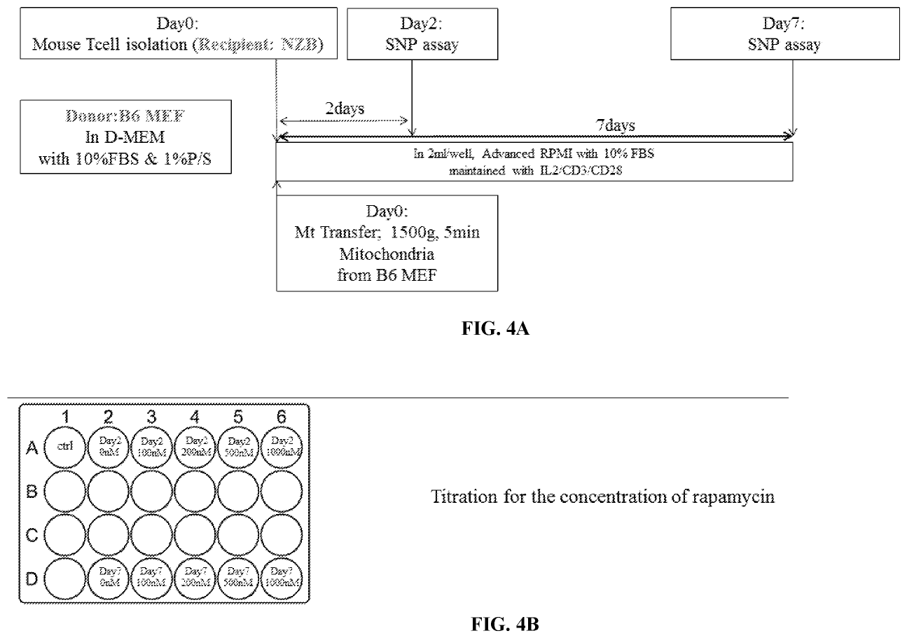

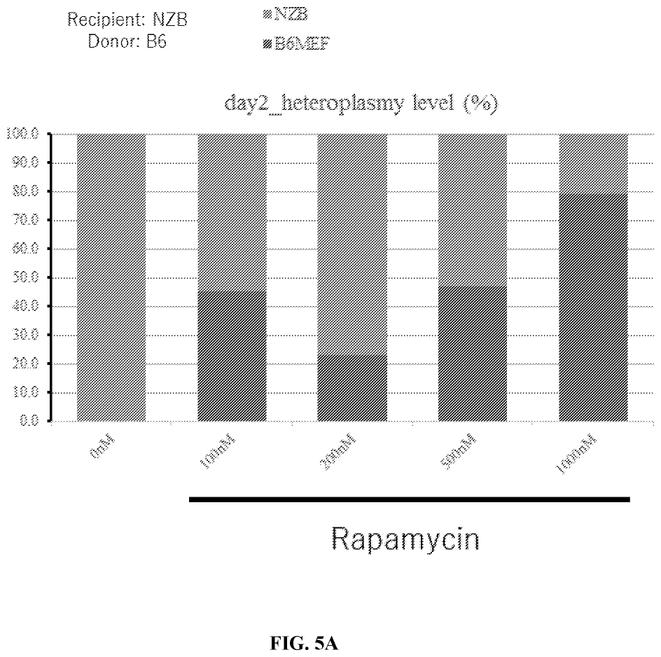

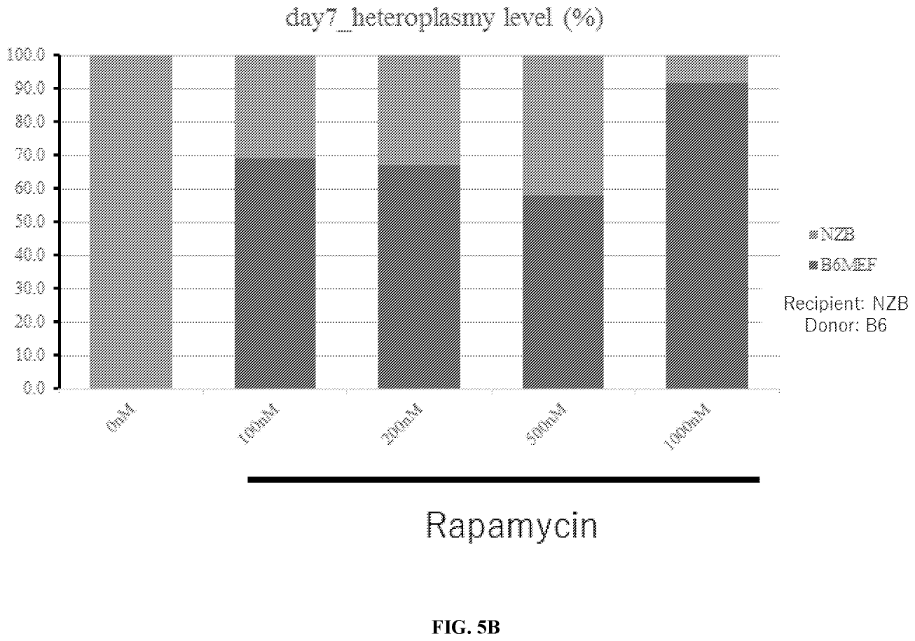

On days 2 and 7 after culturing the cells with the B6 MEF mitochondria in the presence of absence of rapamycin, SNP assays were conducted to detect the replacement of NZB T cell mtDNA with B6 MEF mtDNA. See FIG. 4A for a scheme showing the protocol and FIG. 4B for a depiction of the titration of rapamycin in the wells. As shown in FIG. 5A and FIG. 5B, an increase in the replacement of NZB T cell mtDNA was detected following culture with rapamycin, whereas no replacement of NZB T cell mtDNA was detected without rapamycin. For example, following centrifugation and two days of rapamycin treatment, there was between about 45-80% of donor mtDNA in the recipient T cells (FIG. 5A). By day 7, the amount of donor mtDNA in the recipient T cells increased to approximately 70-90% (FIG. 5B). The amount of replacement of NZB T cell mtDNA with B6 MEF mtDNA increased in a concentration dependent manner. In addition, the amount of B6 MEF mtDNA in NZB T cells on day 7 was higher than that on day 2.

Taken together, the results demonstrate that concomitant rapamycin treatment at the time of contacting the T cells with donor mitochondria can facilitate mitochondrial transfer. Thus, this example demonstrates the successful production of mitochondrial replacement in T cells without reduction of endogenous mtDNA by endonuclease and without genetic manipulation using a protocol involving rapamycin.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method for generating mitochondria replaced lymphoid cells in which at least 20% of endogenous mitochondrial DNA (mtDNA) has been replaced with exogenous mtDNA, wherein the method comprises
incubating lymphoid cells that have not undergone a procedure to reduce or deplete endogenous mitochondria with isolated exogenous mitochondria and an effective amount of mammalian target of rapamycin (mTOR) inhibitor for a sufficient period of time to non-invasively transfer the exogenous mitochondria to the lymphoid cells, thereby generating mitochondria replaced lymphoid cells in which at least 20% of the endogenous mtDNA has been replaced with exogenous mtDNA.

2. The method of claim 1, wherein the mTOR inhibitor comprises rapamycin or a derivative thereof.

3. The method of claim 1 or claim 2, wherein the mTOR inhibitor is rapamycin.

4. The method of any one of claims 1 to 3, wherein the effective amount of the mTOR inhibitor is a concentration of about 100 nM to about 1000 nM.

5. The method of any one of claims 1 to 3, wherein the effective amount of the mTOR inhibitor is a concentration of about 200 nM to about 500 nM.

6. The method of any one of claims 1 to 3, wherein the effective amount of the mTOR inhibitor is a concentration of about 100 nM.

7. The method of any one of claims 1 to 3, wherein the effective amount of the mTOR inhibitor is a concentration of about 200 nM.

8. The method of any one of claims 1 to 3, wherein the effective amount of the mTOR inhibitor is a concentration of about 500 nM.

9. The method of any one of claims 1 to 3, wherein the effective amount of the mTOR inhibitor is a concentration of about 1000 nM.

10. The method of any one of claims 1 to 9, wherein the mitochondria replaced lymphoid cells comprises at least 20% of exogenous mtDNA and no more than 80% endogenous mtDNA, as measured by TaqMan Single Nucleotide Polymorphism (SNP) Assay.

11. A method for generating mitochondria replaced lymphoid cells, wherein the method comprises incubating lymphoid cells that have not undergone a procedure to reduce or deplete endogenous mitochondria with isolated exogenous mitochondria and about 100 nM to about 1000 nM of rapamycin for a sufficient period of time to non-invasively transfer the exogenous mitochondria to the lymphoid cells, thereby generating a mitochondria replaced lymphoid cells.

12. The method of any one of claims 1 to 11, wherein the isolated exogenous mitochondria is about 20 μg to 80 μg protein per 1×10⁶ cells.

13. The method of any one of claims 1 to 12, further comprising centrifuging the lymphoid cells prior to incubating.

14. The method of claim 13, wherein centrifuging is performed at 1,500 relative centrifugal force (RCF) for approximately 5 minutes at room temperature.

15. The method of any one of claims 1 to 14, wherein the sufficient period of time is at least approximately 24 hours.

16. The method of any one of claims 1 to 14, wherein the sufficient period of time is at least 36 hours.

17. The method of any one of claims 1 to 14, wherein the sufficient period of time is at least 48 hours.

18. The method of any one of claims 1 to 14, wherein the sufficient period of time is approximately 2 days or more.

19. The method of any one of claims 1 to 14, wherein the sufficient period of time is approximately 7 days or more.

20. The method of any one of claims 1 to 14, wherein the sufficient period of time is approximately 2 days to approximately 7 days.

21. A method for generating mitochondria replaced lymphoid cells, wherein the method comprises:
(a) centrifuging lymphoid cells and isolated exogenous mitochondria under conditions sufficient to generate a cell pellet, wherein the lymphoid cells have not undergone a procedure to reduce or deplete endogenous mitochondria; and
(b) incubating the lymphoid cells with 100 nM to 1000 nM of rapamycin for approximately 24 hours or more, thereby generating mitochondria replaced lymphoid cells.

22. The method of claim 21, wherein incubating is for approximately 7 days or more.

23. The method of claim 21, wherein incubating is for approximately 2 days to approximately 7 days.

24. The method of any one of claims 1 to 23, wherein the lymphoid cells are T cells, B cells, monocytes, macrophages, natural killer (NK) cells, or granulocytes.

25. The method of claim 24, wherein the lymphoid cells are T cells.

26. The method of claim 25, wherein the T cells comprise exhausted T cells, senescent T cells, or a combination thereof.

27. The method of any one of claims 1 to 26, wherein the lymphoid cells are human lymphoid cells.

28. A composition comprising an effective amount of the mitochondria replaced lymphoid cells generated by the method of any one of claims 1 to 27, and a pharmaceutically acceptable carrier.

29. A method for ameliorating a symptom of mitochondrial complex III deficiency to a subject in need thereof, comprising administering to the subject the composition of claim 28.

30. A method for treating an immunological deficiency associated with heteroplasmic immune cells to a subject in need thereof, comprising administering to the subject the composition of claim 28.

31. The method of claim 30, wherein the subject has received a reverse transcriptase inhibitor.

32. The method of claim 31, wherein the subject has human immunodeficiency virus (HIV).

33. The method of claim 31, wherein the subject has hepatitis B virus (HBV).

34. The method of any one of claims 29 to 33, wherein the subject is human.


SEQUENCE LISTING (13 sequences)

SEQ ID NO: 1 - DNA, 15 bp, Artificial Sequence
  Human T cell-probe
caaccaaccctcaac

SEQ ID NO: 2 - DNA, 17 bp, Artificial Sequence
  EPC100-probe
agcaatcaaccttcaac

SEQ ID NO: 3 - DNA, 22 bp, Artificial Sequence
  NZB mouse probe
tgttctttattaatgccctaac

SEQ ID NO: 4 - DNA, 23 bp, Artificial Sequence
  B6 mouse probe
tcttattaatatcctaacactcc

SEQ ID NO: 5 - DNA, 22 bp, Artificial Sequence
  Human HVR1-F primer
ccccatgcttacaagcaagtac

SEQ ID NO: 6 - DNA, 22 bp, Artificial Sequence
  Human HVR1-R primer
ttggagttgcagttgatgtgtg

SEQ ID NO: 7 - DNA, 23 bp, Artificial Sequence
  Mouse ND1-F primer
ccttgttcccagaggttcaaatc

SEQ ID NO: 8 - DNA, 20 bp, Artificial Sequence
  Mouse ND1-R primer
tgccgtatggaccaacaatg

SEQ ID NO: 9 - DNA, 103 bp, Artificial Sequence
  Human GJ T cell HVR1
cccccccccatgcttacaagcaagtacagcaaccaaccctcaactatcacacatcaactgcaactccaaagccacctctcacccactaggataccaacaaacc

SEQ ID NO: 10 - DNA, 239 bp, Artificial Sequence
  EPC100 HVR1
gaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccttcaactatcacacatcaactgcaactccaaagccacccctcacccactagg

SEQ ID NO: 11 - DNA, 22 bp, Artificial Sequence
  hmtD-Loop-F primer
ctctgttctttcatggggaagc

SEQ ID NO: 12 - DNA, 21 bp, Artificial Sequence
  hmtHV1-R primer
atccatggggacgagaaggga

SEQ ID NO: 13 - DNA, 400 bp, Artificial Sequence
  D-Loop sequence of human mtDNA
attctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgac